(19) United States
(12) Patent Application Publication
(10) Pub. No.: US 2022/0152080 A1
(43) Pub. Date: May 19, 2022

(54) RSK INHIBITORS IN THE TREATMENT OF VIRUS DISEASES

(71) Applicant: Atriva Therapeutics GmbH, Tübingen (DE)

(72) Inventors: Stephan Ludwig, Münster (DE); Oliver Planz, Dettingen an der Erms (DE)

(21) Appl. No.: 17/440,139

(22) PCT Filed: Mar. 19, 2020
(86) PCT No.: PCT/EP2020/057621

§ 371 Date: Sep. 16, 2021

(30) Foreign Application Priority Data
Mar. 19, 2019 (LU) LU101156

(51) Int. Cl. A61K 31/713; A61K 31/496; A61K 31/5375; A61K 31/5517; A61K 31/702; A61K 31/4188; A61K 45/06; A61P 31/12; C12N 15/113

(52) U.S. Cl. CPC A61K 31/713; A61K 31/496; A61K 31/5375; A61K 31/5517; A61K 31/702; C12Q 1/485; A61K 45/06; A61P 31/12; C12N 15/1137; C12N 2310/14; C12Y 207/11001; A61K 31/4188

(57) ABSTRACT

The present invention relates to RSK inhibitors that are capable of displaying one or more beneficial therapeutic effects. The RSK inhibitors can be used in the prevention and/or treatment of viral infection. RSK inhibitors alone or in combination with other anti-viral inhibitor compounds are capable of displaying one or more beneficial therapeutic effects in the treatment of viral diseases.

### FIELD OF THE INVENTION

The present invention relates to RSK inhibitors that are capable of displaying one or more beneficial therapeutic effects. The RSK inhibitors can be used in the prevention and/or treatment of viral infection. RSK inhibitors in combination with other antiviral compounds are capable of displaying one or more beneficial therapeutic effects in the treatment of viral diseases.

### BACKGROUND OF THE INVENTION

Infections with viruses are a significant threat for the health of man and animal. For instance, infections with influenza viruses still belong to the big epidemics of mankind and cause a large number of casualties year for year.

The problem of controlling in particular RNA viruses is the adaptability of the viruses caused by a high error rate of the viral polymerases, which makes the production of suitable vaccines as well as the development of antiviral substances difficult. Furthermore it has been found that the use of antivirals directed against the functions of the virus will sooner or later lead to the selection of resistant variants based on mutation. An example is the anti-influenza agent amantadine and its derivatives directed against a transmembrane protein of the virus. Within a short time after the application, resistant variants of the virus are generated. Other examples are antivirals for influenza infections inhibiting the influenza-viral surface protein neuraminidase, such as oseltamivir, where resistant variants have also broadly emerged in the past.

Because of the very small genome and thus limited coding capacity for functions being necessary for replication, all viruses are dependent to a high degree on functions of their host cells. By exertion of influence on such cellular functions being necessary for viral replication, it is possible to negatively affect virus replication in the infected cell. Herein, there is no possibility for the virus to replace the lacking cellular function by adaptation, in particular by mutations, in order to thus escape from the selection pressure. This could already be shown for the influenza A virus with relatively unspecific inhibitors against cellular kinases and methyl transferases (Scholtissek and Müller, Arch Virol 119, 111-118, 1991).

Like any other virus, Influenza viruses capture the infected cells to replicate. Viral proteins are not only interacting with themselves but with cellular components. Thus, blockage of such constituents might not only inhibit the replication on a broad anti-viral level but could also decrease the occurrence of resistant virus variants due to the inability of the virus to substitute missing cellular functions (Ludwig, 2003) (Ludwig, 2011).

During the influenza viral life cycle newly synthesized ribonucleoprotein complexes (vRNP), containing the viral genome, must be exported from the nucleus, where viral genome replication occurs, to the cytoplasm to be transported to the cellular membrane and packaged in progeny viruses, which are released from the infected cells. The RNPs are exported out of the nucleus via the Crm1-mediated nuclear export pathway, as cytoplasmic accumulation is blocked by the CRM1-inhibitor leptomycin B (Elton, 2001) (Watanabe, 2001). The organization of the vRNP nuclear export complex is to date not fully understood. One putative model postulates the interaction of the nuclear export protein (NEP) with the viral polymerase complex to create a supporting binding site for the matrix protein 1. The Crm1-interaction is mediated via the NEP N-terminus (Brunotte, 2014). Taken into account that the vRNP export does not take place in the absence of M1 but strongly reduced amounts of NEP do not influence this process, the exact contributions of M1 and NEP to the RNP export remains elusive (Wolstenholme, 1980) (Smith, 1985) (Martin, 1991) (Bui, 2000). It was shown that the RNP export complex assembles at the dense chromatin to gain access to the cellular export machinery. This assembly takes place within RCC1 (Ran nucleotide exchange factor)-located regions, to ensure the direct interaction of the RNPs with regenerated Crm1-RanGTP-complexes (Nemergut, 2001) (Chase, 2011).

Furthermore, the virus needs to activate the Raf/MEK/ERK signal transduction pathway to ensure the successful nuclear export (Pleschka, 2001) (Ludwig, 2004). This signaling cascade is a member of the classical mitogen-activated protein kinase (MAPK) cascades and regulates proliferation, differentiation and cell survival (Lewis, 1998) (Yoon, 2006). Influenza virus infection triggers the pathway activation in a biphasic manner, very early after the infection and in the later phase. MEK-specific inhibitors not only suppress both activation phases but in addition lead to strongly decreased viral titers caused by the nuclear retention of newly synthesized RNPs. These effects are shown for influenza A and B viruses (Pleschka, 2001) (Ludwig, 2004) (Haasbach, 2017). No escape mutants could be found after the use of the MEK-inhibitors, such as U0126, in contrast to amantadine treatment (Ludwig, 2004). In addition, Oseltamivir resistant influenza strains can still be inhibited with a MEK-inhibitor treatment (Haasbach, 2017). Furthermore, it was recently published that the inhibition of the pathway reduces viral titers after the infection with the respiratory syncytial virus (Preugschas, 2018). These findings indicate the inability of the viruses to compensate for the missing cellular function and enable a novel antiviral strategy. To date, the exact mechanisms how this pathway triggers the influenza RNP export is unknown.

As mentioned above, influenza viruses exploit the cellular Raf/MEK/ERK signaling pathway to support the export of newly synthesized genomes (in the form of RNA-protein complexes called vRNP) from the nucleus of the infected cell. In previous studies, it was shown that inhibitors of MEK, the central kinase of the Raf/MEK/ERK pathway, block virus replication in vitro and in vivo without side effects and a high barrier towards development of resistance (WO 2014/056894).

However, the direct link between the activated Raf/MEK/ERK pathway to the export of vRNP complexes remained unclear. In general, the Raf/MEK/ERK pathway has been studied for its role in cancer, and it is known that ERK and RSK act downstream of MEK. However, in the prior art it was found that Rsk inhibition, in particular Rsk2 knock-down, exerts a virus supportive effect (Kakugawa et al. (2009) J Virol.; 83(6):2510-7).

Nevertheless, in view of the prior art, it is clear that there is the need of further compounds and compositions effective in the treatment of virus diseases in particular in diseases caused by influenza virus.

### SUMMARY OF THE INVENTION

The present invention relates to an RSK inhibitor for use in a method for the prophylaxis and/or treatment of a viral disease. Specifically, the RSK inhibitor can be selected from the group consisting of BI-D1870, SL0101-1, LJH685, LJ1308, BIX 02565, FMK and a selective RSK1 inhibitor or a derivative, metabolite or pharmaceutically acceptable salt thereof. The selective RSK1 inhibitor can be a si-RNA, shRNA or mi-RNA that selectively targets Rsk1 mRNA corresponding to conserved regions of RSK1 having the amino acid sequence of SEQ ID NO:1 or an antibody that binds to conserved regions of RSK1 having the amino acid sequence of SEQ ID NO:1.

The viral disease can be an infection caused by a positive or negative strand RNA virus. In a preferred embodiment, the viral disease caused by negative strand RNA virus is an influenza virus, such as influenza virus is of type H1N1, H2N2, H3N2, H5N1, H5N6, H5N8, H6N1, H7N2, H7N7, H7N9, H9N2, H10N7, N10N8 or H5N1, or a different influenza A virus or influenza B virus such as the Yamagata or Victoria type. In some embodiments, the influenza virus is resistant to a neuramidase inhibitor selected from the group consisting of oseltamivir, oseltamivir phosphate, zanamivir, peramivir, or laninamivir or a pharmaceutically acceptable salt thereof, or an inhibitor of the viral polymerase complex selected form the group of Favipiravir, Baloxavir, Baloxavir phosphate, Baloxavir Marboxil, or Pimodivir or a pharmaceutically acceptable salt thereof.

In another preferred embodiment, the viral disease caused by a positive strand RNA virus is a coronavirus causing a respiratory tract infection, such as SARS, MERS or Covid-19.

In some cases, the RSK inhibitor is administered in combination with a second antiviral agent, such as a neuramidase inhibitor, polymerase complex inhibitor, endonuclease inhibitor, hemaglutinin inhibitor, non-structural protein 1 inhibitor, nucleoprotein inhibitor or a MEK inhibitor.

The neuramidase inhibitor can be oseltamivir, oseltamivir phosphate, zanamivir, peramivir or laninamivir or a pharmaceutically acceptable salt thereof.

The polymerase complex inhibitor can be Baloxavir, Baloxavir phosphate, Baloxavir Marboxil, Favipiravir or Pimodivir or a pharmaceutically acceptable salt thereof.

The MEK inhibitor can be CI-1040, PD-0184264, PLX-4032, AZD6244, AZD8330, AS-703026, GSK-1120212, RDEA-119, RO-5126766, RO-4987655, PD-0325901, GDC-0973, TAK-733, PD98059 or PD184352, or a pharmaceutically acceptable salt thereof.

The invention also relates to a selective RSK1 inhibitor or a derivative, metabolite or pharmaceutically acceptable salt thereof that binds to RSK1 having the amino acid sequence of SEQ ID NO: 1 or the nucleotide sequence of SEQ ID NO:2 and shows no or low binding affinity to RSK2 having the amino acid sequence of SEQ ID NO: 3 or the nucleotide sequence of SEQ ID NO:4, RSK3 having the amino acid sequence of SEQ ID NO: 5 or the nucleotide sequence of SEQ ID NO:6 and RSK4 having the amino acid sequence of SEQ ID NO: 7 or the nucleotide sequence of SEQ ID NO:8. The selective RSK1 inhibitor can be an si-RNA, shRNA, mi-RNA, an antibody, or a small molecule. The RSK1 inhibitor can be used in a pharmaceutical composition alone or in combination with a second antiviral agent as defined above for the prophylaxis and/or treatment of viral disease.

In addition, the invention relates to a method of identifying specific RSK1 inhibitors comprising the steps of: Screening a library of potential inhibitors for binding to RSK1 having the amino acid sequence of SEQ ID NO: 1 or the nucleotide sequence of SEQ ID NO:2, selecting the inhibitors that were found to bind RSK1 and screening these for binding to RSK2 having the amino acid sequence of SEQ ID NO: 3 or the nucleotide sequence of SEQ ID NO:4, RSK3 having the amino acid sequence of SEQ ID NO: 5 or the nucleotide sequence of SEQ ID NO:6 and RSK4 having the amino acid sequence of SEQ ID NO: 7 or the nucleotide sequence of SEQ ID NO:8; and selecting the inhibitors from step (ii) that do not bind to RSK2, RSK3 or RSK4 as specific RSK1 inhibitors.

Definitions

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

As used herein, the term “RSK” refers to Human RSK (p90 ribosomal S6 kinase), which is present in four different isoforms, termed RSK1 (SEQ ID NO:1 and 2), RSK2 (SEQ ID NO:3 and 4), RSK3 (SEQ ID NO:5 and 6) and RSK4 (SEQ ID NO:7 and 8). An alignment of the four isoforms shown as FIG. 1 on an amino acid level and FIG. 2 on a nucleotide level reveals that these four forms are highly conserved but also have specific regions of diversity.

As used herein, the term “RSK inhibitor” refers to an agent that inhibits RSK. The agent can either inhibit all four RSK isoforms or at least RSK1. The RSK inhibitor can be selected from the group consisting of BI-D1870, SL0101-1, LJH685, LJ1308, BIX 02565, FMK and a selective RSK1 inhibitor or a derivative, metabolite or pharmaceutically acceptable salt thereof. The selective RSK1 inhibitor can be a si-RNA, shRNA or mi-RNA that selectively targets Rsk1 mRNA corresponding to conserved regions of RSK1 having the amino acid sequence of SEQ ID NO:1 or an antibody that binds to conserved regions of RSK1 having the amino acid sequence of SEQ ID NO:1.

As used herein, the term “pharmaceutically acceptable salts, derivatives or metabolites” refers to the relatively non-toxic, organic or inorganic salts of the active compounds, including inorganic or organic acid addition salts of the compound, as well as derivatives and metabolites of the RSK inhibitors claimed that show the same function as RSK inhibitors as the named inhibitors.

As such, the term “treating” or “treatment” includes administration of an RSK inhibitor preferably in the form of a medicament, to a subject suffering from a viral disease for the purpose of ameliorating or improving symptoms accompanying such infections.

Furthermore, the term “prophylaxis” as used herein, refers to any medical or public health procedure whose purpose is to prevent a medical condition described herein. As used herein, the terms “prevent”, “prevention” and “preventing” refer to the reduction in the risk of acquiring or developing a given condition, namely a viral disease as described herein. Also meant by “prophylaxis” is the reduction or inhibition of the recurrence of a viral disease in a subject.

As used herein the term “viral disease” includes disease caused by a virus, for example diseases caused by a positive or negative RNA strand virus. For example influenza viruses are negative RNA strand viruses; for example influenza A and B virus. The influenza virus or influenza virus strain according to the invention may show or have developed a resistance to one or more neuraminidase inhibitors (e.g. oseltamivir, oseltamivir phosphate, zanamivir or peramivir) or the influenza virus or influenza virus strain according to the invention does show or does not have developed a resistance to one or more neuraminidase inhibitors (e.g. oseltamivir, oseltamivir phosphate, zanamivir or peramivir). Influenza virus can be an influenza A virus or influenza B virus, preferably the influenza A virus is H1N1, H2N2, H3N2, H5N1, H5N6, H5N8, H6N1, H7N2, H7N7, H7N9, H9N2, H10N7, N10N8 or H5N1. In some embodiments, the influenza virus is resistant to a neuramidase inhibitor selected from the group consisting of oseltamivir, oseltamivir phosphate, zanamivir, peramivir, or laninamivir or a pharmaceutically acceptable salt thereof, or an inhibitor of the viral polymerase complex selected form the group of Favipiravir, Baloxavir, Baloxavir phosphate, Baloxavir Marboxil, or Pimodivir or a pharmaceutically acceptable salt thereof.

Positive strand RNA viruses can be, for example, Coronaviruses such as SARS-CoV, MERS-CoV or SARS-CoV-2 that cause respiratory tract infections such as SARS, MERS or Covid19.

As used herein the term “second antiviral agent” refers to a known antiviral selected from the group comprising neuramidase inhibitors, polymerase complex inhibitors, endonuclease inhibitor, hemaglutinin inhibitors, nucleoprotein inhibitors and a MEK inhibitors.

A “neuraminidase inhibitor” is an antiviral drug targeted at influenza virus, which works by blocking the function of the viral neuraminidase protein, thus preventing virus from being released from the infected host cells, since the newly produced viruses cannot bud off from the cell in which they have replicated. Also comprised are pharmaceutically acceptable salts of a neuraminidase inhibitor. Preferred neuraminidase inhibitors are oseltamivir, zanamivir, peramivir, laninamivir or a pharmaceutically acceptable salt of any of these substances, such as oseltamivir phosphate, oseltamivir carboxylate, etc. The neuramidase inhibitors can be oseltamivir, oseltamivir phosphate, zanamivir, peramivir or laninamivir or a pharmaceutically acceptable salt thereof. Most preferred neuraminidase inhibitors are oseltamivir phosphate, zanamivir, oseltamivir or peramivir

Compounds targeting polymerase or endonuclease activity via interfering with a component of the viral polymerase complex, PB1, PB2, PA or NP are for example the NP blocker nucleozin or the polymerase inhibitor T-705 (Favipiravir). The preferred “polymerase complex inhibitors” are Baloxavir, Baloxavir phosphate, Baloxavir Marboxil, Favipiravir or Pimodivir or a pharmaceutically acceptable salt thereof.

“MEK inhibitors” inhibit the mitogenic signaling kinase cascade Raf/MEK/ERK in cells or in a subject by inhibiting the MEK (mitogen-activated protein kinase kinase). This signaling cascade is hijacked by many viruses, in particular influenza viruses, to boost viral replication. Specific blockade of the Raf/MEK/ERK pathway at the bottleneck MEK therefore impairs growth of viruses, in particular influenza viruses. Additionally, MEK inhibitors show low toxicity and little adverse side effects in humans. There is also no tendency to induce viral resistance (Ludwig, 2009). The MEK inhibitors can be CI-1040, PD-0184264, PLX-4032, AZD6244, AZD8330, AS-703026, GSK-1120212, RDEA-119, RO-5126766, RO-4987655, PD-0325901, GDC-0973, TAK-733, PD98059 or PD184352, or a pharmaceutically acceptable salt thereof. A combination with CI-1040 or PD-0184264 is considered as one specific embodiment.

### DETAILED DESCRIPTION OF THE INVENTION

As mentioned above, it is known that viral infection leads to the activation of a variety of signaling processes in the infected cells. Some of these activities are necessary for an efficient viral replication. The dependence of the viruses, specifically the Influenza A virus (IAV), on cellular signaling pathways leads to the opportunity of a novel antiviral strategy by targeting host factors that are essential for viral replication. IAV infection induces the Raf/MEK/ERK kinase cascade for an efficient nuclear export of newly synthesized viral ribonucleoproteins (vRNP) and this mechanism can be blocked with specific MEK-inhibitors. Such antiviral strategies reduce the possibility of inducing viral resistance and enable a larger timeframe for a further antiviral treatment. However, the detailed mechanism how this cellular kinase cascade contributes to the nuclear export of the viral genome is still enigmatic. For this reason, the role of the Raf/MEK/ERK/RSK signaling pathway in the interaction of vRNPs with the viral M1 protein at the chromatin by using specific MEK-inhibitors, such as CI-1040 (Haasbach et al., (2017) Antiviral. Res. 2017 142:178-184) and RSK-inhibitors, such as BI-D1870, was studied in the present invention.

The invention is based on the object to provide substances for use in the prevention or treatment of viral diseases, in particular prevention and/or therapy against intracellular and/or intranuclear-replicating negative strand RNA viruses, such substances not being immediately directed against functions of the virus, but selectively inhibiting a cellular enzyme, and inhibiting via this selective effect the viral replication of viruses.

In the studies leading to the present invention, the molecular mode of action of RSK was analysed in view of the interaction of vRNPs with the viral matrix protein (M1) at the chromatin by using specific inhibitors against MEK (CI-1040) and RSK (BI-D1870), as well as siRNAs against ERK and RSK. To gain insights into nuclear distribution of viral proteins, chromatin fractionation assays and stochastic optical reconstruction microscopy (STORM) was used, as can be seen from the Examples. To analyze putative phosphorylation targets of the pathway in the viral proteins vRNPs were purified after inhibitor treatment and their phosphorylation patterns were analyzed via mass spectrometry.

It could be shown that the inhibition of the pathway specifically blocks the export of the vRNPs and does not generally influence CRM1 dependent nuclear export of other cellular proteins, as exemplified by undisturbed export of RanBP1. This indicates that viral proteins in the vRNPs may be modified via the Raf/MEK/ERK pathway to promote nuclear export. the location of the vRNP complexes within the nuclei of infected and inhibited cells was investigated and a retention at the chromatin was found. Immunepurification of the vRNP complexes revealed a reduced binding ability to the M1 protein in the presence of the kinase inhibitors. These results could be explained by a missing phosphorylation of the two serine residues 269 and 392 within the viral nucleoprotein (NP), which under normal conditions might induce the binding of the M1 protein to the vRNPs. It was further shown that the kinase RSK, a downstream effector of the Raf/MEK/ERK pathway is a mediator of the export-enhancing function. The inhibition of RSK led to titer reductions due to nuclear retention of vRNPs for all tested influenza viruses, including the pandemic 2009 swine flu H1N1, the highly pathogenic bird flu viruses H5N1 and H7N9, as well as influenza B virus.

These results indicate that the Raf/MEK/ERK pathway is activated by influenza viruses to successfully assemble the vRNP nuclear export complex at the chromatin by RSK-dependent phosphorylation of the NP protein and providing an interaction site for the viral M1 protein.

In Example 1 it was shown that the Raf/MEK/ERK-pathway is dependent on phosphorylation of a specific motif within the nucleoprotein. Under physiological conditions the Raf/MEK/ERK kinase cascade transmits extracellular signals within the cell to promote cellular processes like proliferation and differentiation. The signal is transmitted via sequential phosphorylation of the protein kinases (Yoon, 2006). As shown in FIG. 3, it was found that two Serine residues showed a decreased phosphorylation state after inhibition of the pathway with MEK inhibitor CI-1040. The crystallographic structure of a vRNP with bound RNA revealed that the two Serine residues 269 and 392 are in close proximity to each other. As can be seen from FIG. 3B, the identified regions within the NP protein (LILRGS²⁶⁹V, AIRTRS³⁹²G) showed no similarity to the consensus sequence of the Serine/Threonine-kinase ERK (Pro-Xaa-Ser/Thr-Pro) ((Gonzalez, 1991). Therefore it is unlikely that the identified Serine residues are directly phosphorylated by the ERK kinase. The consensus sequences of the downstream kinase 90 kDa ribosomal S6 kinase (RSK) (Arg/Lys-Xaa_Arg-Xaa-Xaa-Ser/Thr; Arg-Arg-Xaa-Ser/Thr) showed higher identities to the identified NP regions (Romeo, 2012).

In Example 2, it is demonstrated that RSK1 acts as a link between the Raf/MEK/ERK-pathway and vRNP export. To shed light on the question whether RSK is the link between the activation of the Raf/MEK/ERK signaling pathway and the nuclear export of newly synthesized vRNPs, its activation during the viral life cycle was analyzed. In the later stages of the infection not only ERK but also RSK and its downstream target GSK-3ß were phosphorylated and activated (FIG. 4A). This activation could be blocked by incubation with the RSK inhibitor BI-D1870, indicating that the virus induced RSK activation is mediated by the Raf/MEK/ERK pathway (FIG. 4B). In addition, there were no significant differences in progeny viral titers between the CI-1040 treatment and the BI-D1870 and CI-1040 combinational treatment, further showing that RSK directly gets activated by the virus induced Raf/MEK/ERK pathway (FIG. 4J). The inhibition of RSK with the specific inhibitor BI-D1870 after virus infection led to a concentration dependent reduction of the GSK-3ß phosphorylation, confirming its inhibitory effect on the RSK activation during the viral life cycle. Furthermore, we found an increase of the ERK activation after the inhibition of RSK, which can be explained by the inhibition of a negative feedback loop that under normal conditions would prevent the overactivation of the pathway (FIG. 4K). Increasing concentrations of the inhibitor BI-D1870 negatively affected the vRNP nuclear export.

In addition, to exclude off target effects of the RSK inhibitors, RSK1 and RSK2 knockdowns were introduced in A549 cells and the effect on the viral life cycle were analyzed (FIG. 4). The RSK1 knockdown led to a nuclear retention of newly synthesized vRNPs whereas the RSK2 knockdown had no effect on the nuclear export (FIG. 4F-I). Within the multi replication analysis the RSK1 knockdown had an antiviral effect, shown by the decrease of viral titers. The RSK2 knockdown however seemed to have a proviral effect, as shown in FIG. 5. This supportive effect of the RSK2 knockdown on the viral replication was already described by Kakugawa et al., 2009. This points out that the two RSK subtypes RSK1 and RSK2 have different roles within the influenza virus life cycle.

Further, in Example 3, it was shown that MEK- and RSK-inhibitors specifically block vRNP export, without interfering with the export of other proteins. Unlike Leptomycin B, which very specifically blocks the nuclear export pathway, RSK and MEK inhibitors were found to have no general effect on the nuclear export pathway but are more specific against the export of the vRNPs as shown in FIG. 6.

Finally, in Example 4 it was found that the RSK inhibitor BI-D1870 has a broad anti-influenza activity. Here different influenza A subtypes, including a swine origin pandemic virus and avian influenza viruses, and an influenza B virus to determine the broad anti-viral activity. All tested viruses showed a nuclear retention of newly synthesized vRNPs and a reduction of progeny viral titers of around 80 to 90%. These results clarify the dependence of the influenza virus on the Raf/MEK/ERK/RSK pathway (FIG. 7) and the role of RSK as an effector in this cascade.

From these experiments, it was concluded that the kinase Rsk, particularly the isoform Rsk1 (SEQ ID NO:1 and 2), is the downstream mediator of the Raf/MEK/ERK pathway and most likely directly phosphorylates the viral nucleoprotein, which is the major constituent of the vRNP complexes. This posttranslational modification appears to be required for the vRNP export. Thus inhibitors of Rsk have antiviral activity against viruses by preventing export of viral genomes from the nucleus. This is an unexpected finding, because preexisting literature says that Rsk inhibition, in particular Rsk2 knock-down, exerts a virus supportive effect (Kakugawa et al. (2009) J Virol.; 83(6):2510-7). Surprisingly, it has been found that this object can be achieved by an RSK inhibitor according to the invention, in particular by pharmaceutical compositions comprising a RSK1 inhibitor compound.

As mentioned above, human RSK (p90 ribosomal S6 kinase) is present in four different isoforms, termed RSK1, RSK2, RSK3 and RSK4. An alignment of the four isoforms shown as FIG. 1 on an amino acid level and FIG. 2 on a nucleotide level reveals that these four forms are highly conserved but also have specific regions of diversity. While RSK family members are generally viewed as multifunctional effectors of the Ras/MAPK pathway, individual isoforms appear to possess both overlapping and specific functions. While RSK1 and RSK2 play roles in cell growth and proliferation, RSK3 and RSK4 have been associated to cell cycle arrest and apoptosis. In the present invention it was found that inhibition of RSK1 alone has an anti-viral effect, while inhibition of RSK2 alone has a pro-viral effect. However, when a general RSK inhibitor, such as BI-D1870 or SL0101 is used, the inhibition has an antiviral effect, so it seems that the pro-viral activity of RSK1 is dominant over the anti-viral effect of RSK2. This is in line with the experimental evidence that only RSK1, not RSK2 is involved in the nuclear export of vRNPs. Thus both a specific RSK1 inhibitor and a general RSK inhibitor have an anti-viral effect.

These experiments revealed different contributions of the isoforms RSK1 and RSK2 to the viral life cycle. RSK2 has an anti-viral effect, whereas RSK1 obviously seems to act virus supportive. Upon its activation at the plasma membrane and the cytosol, RSK1 translocates into the nucleus. If RSK1 is the kinase phosphorylating NP, its cellular distribution should change during the viral life cycle, depending on the virus induced activation of the Raf/MEK/ERK pathway. To address this hypothesis, as described in Example 5, A549 cells were either mock infected or infected with the WSN/H1N1 virus and the localization of NP and RSK1 was analyzed by immunofluorescence staining. As expected, in the later time points of the infection an increase in the nuclear localization of RSK1 can be seen (FIG. 8A). The quantification of three independent experiments revealed a significant change in the nuclear concentration of RSK1 from 35.58%±0.59% for the mock infection to 57.73%±1.47% for the virus infection after 9 h (FIG. 8B), especially when the NP export took place (FIGS. 8A,C). Indicating that the virus induces the nuclear import of RSK1.

After it was confirmed that RSK1 enters the nucleus during the viral life cycle, the question was addressed whether virus induced activation of the Raf/MEK/ERK pathway would result in a nuclear localization of the anti-viral RSK2. Generally, the activation of the Raf/MEK/ERK-pathway results in the translocation of RSK1 and RSK2 into the nucleus. Therefore, the same experiment as described in Example 5 was conducted but the localization of RSK2 was analyzed, as described in Example 6. In contrast to RSK1, no change in the subcellular distribution of RSK2 was found, independently of the time point during the viral infection (FIGS. 9A,B). These results show, that the virus specifically induces the translocation of the virus supportive RSK1 without influencing the cellular distribution of antiviral acting RSK2.

After the specific translocation of RSK1 was discovered, the question did arise whether the RSK upstream kinase ERK1/2 might enter the nucleus due to the viral pathway induction. Surprisingly, as described in Example 7, no effect on the ERK1/2 localization can be seen after viral infection, independently of the analyzed time point (FIGS. 10A,B).

To exclude unspecific staining of RSK2 and ERK1/2 the Raf/MEK/ERK-pathway was stimulated with TPA as described in Example 8. It is known that the incubation with TPA leads to the activation of the Raf/MEK/ERK-pathway resulting in the nuclear localization of ERK1/2 and RSKs within minutes after the stimulation. Both kinases were found in the nuclei after an incubation with 100 nM TPA for 1 h, confirming that the staining is specific (FIGS. 11A,C). The quantification revealed nuclear accumulation of ERK1/2 from 27.99%±1.97% for the unstimulated samples to 48.02%±2.25% for the TPA-stimulated samples. The effect on the RSK2 kinase was not as prominent as for ERK1/2, with 29.37%±1.15% for the unstimulated samples to 34.89%±0.67% for the TPA-stimulated samples (FIGS. 11B,D).

Taken together, during the viral life cycle RSK1 enters the nucleus of the infected cell and acts virus supportive, most likely via phosphorylation of NP. This translocation is mediated by the activation of the Raf/MEK/ERK-pathway. Additionally, the virus appears to prevent nuclear localization of ERK1/2 and the anti-viral RSK2.

A major problem of anti-viral strategies directly targeting the virus particle is the introduction of resistant virus variants. Long-term exposure to such anti-viral drugs provokes the emergence of mutations that lead to a reduced drug susceptibility. One group of anti-viral drugs is represented by the neuraminidase inhibitors, like Oseltamivir. It binds to neuraminidases exposed on the surface of infected cells leading to a conformational change of the active site by creating a binding pocket. The inhibitory effect is reduced by mutations within the neuraminidase, e.g. H275Y, E119D, I223R. A second group are polymerase inhibitors, like Baloxavir. This cap-dependent endonuclease inhibitor blocks the cap-snatching process of the PA subunit. Reduced susceptibility to Baloxavir marboxil can especially occur after the amino acid substitution I38T.

To compare the ability of antiviral drugs targeting the virus particle directly (Oseltamivir, Baloxavir) or acting antiviral via inhibition of the Raf/MEK/ERK/RSK pathway (CI-1040, BI-D1870) to induce resistance virus variants, A549 cells were infected with the WSN/H1N1 virus using a MOI of 0.01 as described in Example 9. Directly after infection, cells were treated with the different inhibitors or DMSO. 24 h p.i. progeny viral titers were determined by standard plaque titration. For the following rounds A549 cells were infected with the virus supernatants (MOI 0.01) of the previous round and titers were determined 24 h p.i. The inhibitor concentrations were increased to enhance the inhibitory effect and provoke the occurrence of mutations. The increasing concentrations of CI-1040 and BI-D1870 resulted in decreasing viral titers. Compared to the DMSO control, the titers were reduced in the first round using a 1 μM inhibitor concentration by 64.75%±0.32% and by 35.08%±3.20% for CI-1040 or BI-D1870, respectively. With increasing inhibitor concentrations, the titers did further reduce, up to round 4. At that point, both inhibitors were used in a concentration of 8 μM. From round 5 to round 12 CI-1040 and BI-D1870 were used with concentrations of 10 μM. Within round 5 to round 12 the average titer of the CI-1040 treatment was calculated with 8.45%±3.41% compared to the DMSO control. The average titer of the BI-D1870 treatment within round 5 to round 12 was calculated with 12.27%±6.36%. Neither a constant increase in the viral titers, nor a change in the plaque morphology was found for any time point during the 12 rounds, indicating that no resistance introducing mutation did occur. A complete resistance was found after 5 rounds of Oseltamivir treatment. At that point, an inhibitor concentration of 16 μM was used. At round 9 the titers started to decrease up to 48.30%±11.42%. This effect was accompanied by an increasing reduction in the plaque size. The average titer of the Baloxavir treatment from round 1 to round 9 was calculated with 8.08%±4.10%. A tendency in the titer increase started at round 10 with an average titer of 28.39%±1.88%. At round 12 the average titer further increased up to 46.94%±1.03% (FIG. 12).

These results support the finding that long-term treatment with the MEK- or RSK-inhibitors does not introduce a resistance in viruses.

These and former results within this study indicate that MEK- and RSK-inhibitors are suitable drugs against virus infections, such as an influenza virus infection. They are well tolerated by the cells, without cytotoxic effects, show a broad anti-viral activity and seem not to promote the occurrence of resistant virus variants

Hence, in an aspect, the present invention provides a method for the prophylaxis and/or treatment of a viral disease comprising administering an RSK inhibitor to a patient in need thereof.

In an aspect, the method of the invention is for the prophylaxis and/or treatment of a viral disease which is an infection caused by negative RNA strand virus. More preferably, the viral disease is caused by an influenza virus, even more preferably is caused by influenza A or B viruses. Influenza A viruses are for example H1N1, H3N2, H5N1, H7N7, H7N9 or H9N2.

In another aspect, the method of the invention is for the prophylaxis and/or treatment of a viral disease which is an infection caused by positive strand RNA virus such as, for example, Coronaviruses such as SARS-CoV, MERS-CoV or SARS-CoV-2 that cause respiratory tract infections such as SARS, MERS or Covid19.

The RSK inhibitors of the invention are selected preferably from BI-D1870, SL0101, LJH685, LJI308, BIX 02565, FMK and a selective RSK1 inhibitor or a derivative, metabolite or pharmaceutically acceptable salt thereof.

Specifically, BI-D1870 is known to be a potent and specific inhibitor of the p90 ribosomal S6 kinase (RSK) isoforms in vitro and in vivo, which inhibits RSK1, RSK2, RSK3 and RSK4 in vitro (IC50 values 31 nM, 24 nM, 18 nM, and 15 nM, respectively). (G P Sapkota et al. BI-D1870 is a specific inhibitor of the p90 RSK (ribosomal S6 kinase) isoforms in vitro and in vivo. Biochem. J. 2007, 401, 29-38).

SL-0101 is a cell-permeable Kaempferol (Cat. No. 420345) glycoside that targets the N-terminal kinase domain of p90 ribosomal S6 kinase and inhibits RSK kinase activity (IC50=89 nM with 10 μM ATP) in a selective, reversible, and ATP-competitive (Ki=1 μM) manner. SL-0101 was shown to inhibit the proliferation of MCF-7, but not the normal breast cell line MCF-10A, and specifically block PDB-induced cellular phosphorylation of RSK substrate p140, but not that of RSK or RSK upstream kinases even at concentrations as high as 100 μM.

All of the known RSK inhibitors listed above inhibit all 4 isoforms of RSK. However, one aim of the present invention is to provide a specific RSK1 inhibitor that is capable of blocking RSK1 without affecting RSK2, RSK3 or RSK4. A method of identifying an RSK1 inhibitor is to perform a screen for substances that bind to RSK1 (SEQ ID NO:1 and 2) but not to RSK2 (SEQ ID NO:3 and 4), RSK3 (SEQ ID NO:5 and 6) or RSK4 (SEQ ID NO:7 and 8).

In the medical uses of the invention, the RSK inhibitor may be administered orally, intravenously, intrapleurally, intramuscularly, topically or via inhalation. Preferably, the RSK inhibitor is administered via nasal inhalation or orally to a subject or patient.

The subject or patient of the invention is a mammal or a bird. Examples of suitable mammals include, but are not limited to, a mouse, a rat, a cow, a goat, a sheep, a pig, a dog, a cat, a horse, a guinea pig, a canine, a hamster, a mink, a seal, a whale, a camel, a chimpanzee, a rhesus monkey and a human. Examples of suitable birds include, but are not limited to, a turkey, a chicken, a goose, a duck, a teal, a mallard, a starling, a Northern pintail, a gull, a swan, a Guinea fowl or water fowl, to name a few. Human patients are a particular embodiment of the present invention.

The pharmaceutical composition comprising the RSK inhibitor may be in the form of orally administrable suspensions or tablets; nasal sprays, sterile injectable preparations (intravenously, intrapleurally, intramuscularly), for example, as sterile injectable aqueous or oleaginous suspensions or suppositories. When administered orally as a suspension, these compositions are prepared according to techniques available in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art. The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

In the treatment methods and medical uses of the invention, the RSK inhibitor is administered in a therapeutically effective amount. The “therapeutically effective amount” can vary with factors including but not limited to the activity of the compound used, stability of the active compound in the patient's body, the severity of the conditions to be alleviated, the total weight of the patient treated, the route of administration, the ease of absorption, distribution, and excretion of the compound by the body, the age and sensitivity of the patient to be treated, adverse events, and the like, as will be apparent to a skilled artisan. The amount of administration can be adjusted as the various factors change over time.

In addition, the RSK inhibitor may be administered together with a second antiviral agent. The second antiviral agent may be administered prior to, concomitantly with or after the administration of the RSK inhibitor. In addition, the RSK inhibitor and the second antiviral agent may be administered in one dosage form or in two separate dosage forms. It is also contemplated that the RSK inhibitor may be administered together with two or more antiviral agents.

The second antiviral agent may be any known antiviral. The second antiviral agent may be selected from the group comprising neuramidase inhibitors, polymerase complex inhibitors, endonuclease inhibitor hemaglutinin inhibitors, nucleoprotein inhibitors and MEK inhibitors.

Preferred neuramidase inhibitors can be oseltamivir, oseltamivir phosphate, zanamivir, peramivir or laninamivir or a pharmaceutically acceptable salt thereof. A combination with oseltamivir is considered as one specific embodiment.

Preferred polymerase complex inhibitors can be Baloxavir, Baloxavir phosphate, Baloxavir Marboxil, Favipiravir or Pimodivir or a pharmaceutically acceptable salt thereof.

Preferred MEK inhibitors can be CI-1040, PD-0184264, PLX-4032, AZD6244, AZD8330, AS-703026, GSK-1120212, RDEA-119, RO-5126766, RO-4987655, PD-0325901, GDC-0973, TAK-733, PD98059 or PD184352, or a pharmaceutically acceptable salt thereof. A combination with CI-1040 or PD-0184264 is considered as one specific embodiment.

### DESCRIPTION OF THE FIGURES

FIG. 1 shows an alignment of RSK1-4 on an amino acid level.

FIG. 2 shows an alignment of RSK1-4 on a nucleotide level.

FIG. 3: shows phosphorylation sites of NP at residues S269 and S392 that are phosphorylated upon Raf/MEK/ERK-activation (A, C) as described in Example 1. In addition, (B) shows a comparison of ERK and RSK consensus sequences. Figure (D) shows the RNA binding sites of the M1 protein with a positively charged Histidine as described in Example 1, and Figure (F) shows the growth kinetics of constitutive non-phosphorylated (NP) mutants versus wild-type (wt), while Figure (G) shows the growth kinetics of M1 Histidine mutants versus wild-type (wt).

FIG. 4(A-H) show decreased M1-NP binding upon RSK-inhibition and nuclear retention of progeny vRNPs upon RSK-inhibition or RSK1 knockdown, as demonstrated in Example 2. (I-P) show that the specific RSK-inhibitors BI-D1870 and SL0101-1 inhibit viral propagation as described in Example 2.

FIG. 5 shows BI-D1870 treatment results in chromatin retention of progeny vRNPs and decreased binding rates with the M1 protein as described in Example 2.

FIGS. 6(A, B) shows that pathway inhibition acts specific against nuclear export of viral proteins, specifically that the Raf/MEK/ERK/RSK-pathway inhibitors CI-1040 and BI-D1870 act specific on the nuclear export of viral proteins without introducing resistant virus variances as described in Example 3. In addition, (C, D) shows a comparison of CI-1040, BI-D1870, Oseltamivir and Baloxavir administered in increasing concentrations.

FIG. 7 shows the broad antiviral effect of RSK-inhibition (A-P) as well as a comparison of the NP regions of different influenza viruses (Q) and the cristallographic structure of Influenza A and B (R).

FIG. 8 shows RSK1 nuclear localization during the WSN/H1N1 infection. (A) A549 cells were infected with WSN/H1N1 (MOI 5) or mock infected. After the indicated time points the cells were fixed and cellular localization of vRNPs (NP-Alexa-488) and RSK1 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. (B, C) Quantification of the cellular localization of NP and RSK1 from (A). 10 epifluorescence microscopy pictures of each sample were analyzed in terms of the NP and RSK1 localization using the ImageJ “Intensity Ratio Nuclei Cytoplasm Tool”. Results are depicted as means±SD of three independent experiments. Statistical significance was analyzed by unpaired t-test with Welch's correction for each time point separately (ns p>0.05; **p≤0.01).

FIG. 9 shows no change in the RSK2 cellular localization during the WSN/H1N1 infection. (A) A549 cells were infected with WSN/H1N1 (MOI 5) or mock infected. After the indicated time points the cells were fixed and cellular localization of vRNPs (NP-Alexa-488) and RSK2 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. (B, C) Quantification of the cellular localization of NP and RSK2 from (A). 10 epifluorescence microscopy pictures of each sample were analyzed in terms of the NP and RSK2 localization using the ImageJ “Intensity Ratio Nuclei Cytoplasm Tool”. Results are depicted as means±SD of three independent experiments. Statistical significance was analyzed by unpaired t-test with Welch's correction for each time point separately (ns p>0.05; **p≤0.05).

FIG. 10 shows no change in the ERK1/2 cellular localization during the WSN/H1N1 infection. (A) A549 cells were infected with WSN/H1N1 (MOI 5) or mock infected. After the indicated time points the cells were fixed and cellular localization of vRNPs (NP-Alexa-488) and ERK1/2 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. (B, C) Quantification of the cellular localization of NP and ERK1/2 from (A). 10 epifluorescence microscopy pictures of each sample were analyzed in terms of the NP and ERK1/2 localization using the ImageJ “Intensity Ratio Nuclei Cytoplasm Tool”. Results are depicted as means±SD of three independent experiments. Statistical significance was analyzed by unpaired t-test with Welch's correction for each time point separately (ns p>0.05).

FIG. 11 shows that stimulation with TPA results in nuclear localization of RSK2 and ERK1/2. (A,C) A549 cells were stimulated with TPA (200 nM). The solvent DMSO served as negative control. After 1 h the cells were fixed and cellular localization of RSK2 or ERK1/2 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm.(B,D) Quantification of the cellular localization of RSK2 or ERK1/2 from (A,C). 15 epifluorescence microscopy pictures of each sample were analyzed in terms of the RSK2 localization using the ImageJ “Intensity Ratio Nuclei Cytoplasm Tool”. Results are depicted as means±SD of three independent experiments. Statistical significance was analyzed by unpaired t-test with Welch's correction (**p≤0.01).

FIG. 12 shows that long-term treatment with the MEK- or RSK-inhibitors CI-1040 or BI-D1870 does not introduce a resistance in WSN/H1N1. (A) A549 cells were infected with WSN/H1N1 (MOI 0.01) and treated with the MEK-inhibitor CI-1040, the RSK-inhibitor BI-D1870, the viral NA-inhibitor Oseltamivir acid or the viral cap-dependent endonuclease inhibitor of the polymerase subunit PA Baloxavir marboxil. 24 h p.i. progeny viral titers were determined by standard plaque titration. During the following rounds fresh A549 cells were infected with the collected supernatants (MOI 0.01) and treated with increasing concentrations of the different inhibitors. The rounds at which the concentrations remained constant are marked by arrowheads. The solvent DMSO (1%) served as negative control. Shown are relative viral titers. Titers of the DMSO control were arbitrary set to 100% (marked by dashed line). Data represents means±SD of two independent experiments. Each experiment was performed in triplicates. Statistical significance was calculated by two-way ANOVA followed by Bonferroni post-test (***p≤0.001). (B) Overview of the inhibitor concentrations used for each round. At round 5 (CI-1040, BI-D1870) or round 6 (Oseltamivir, Baloxavir) the concentrations remained constant.

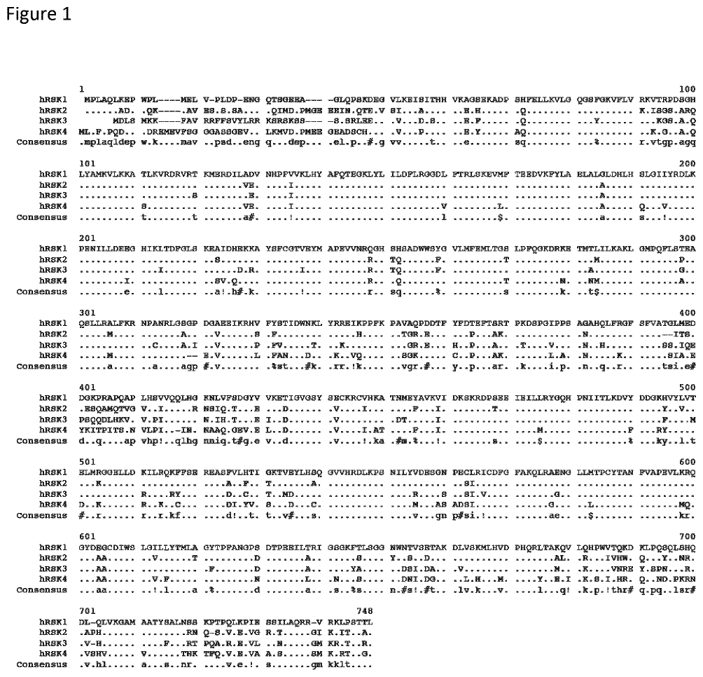

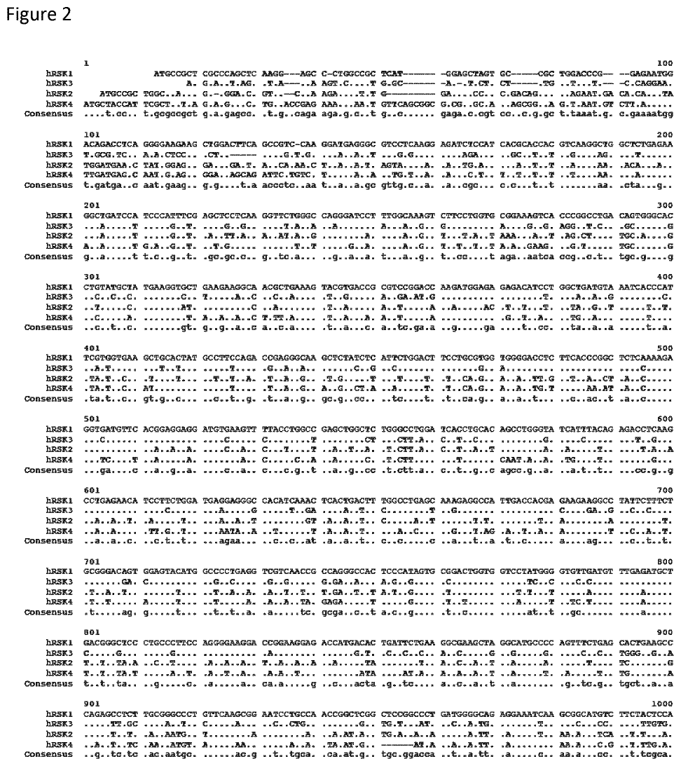

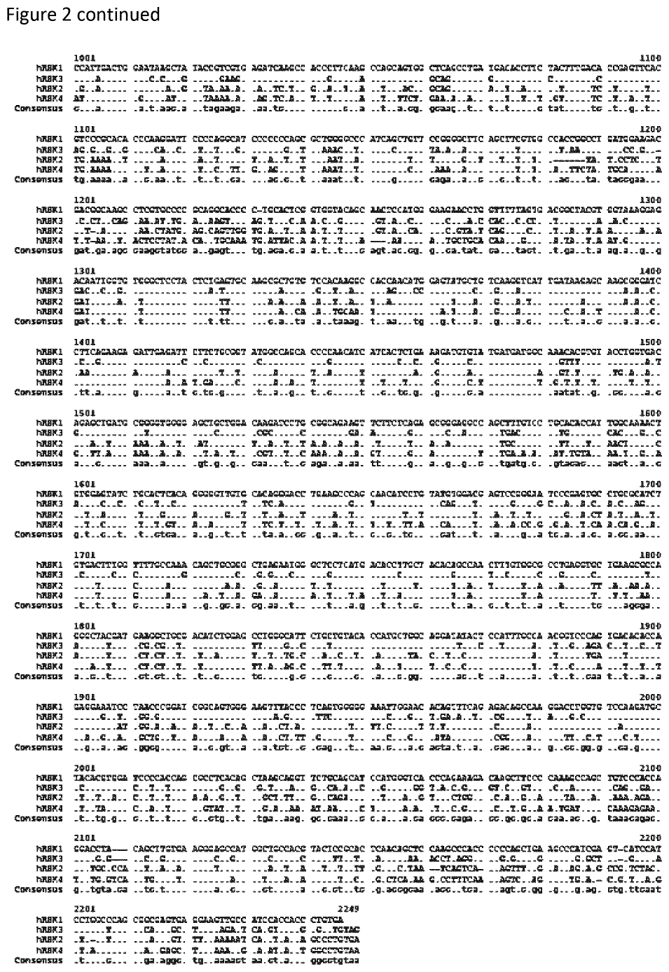

### EXAMPLES

### Example 1

### Raf/MEK/ERK-Pathway Dependent Phosphorylation of a Specific Motif Within the Nucleoprotein

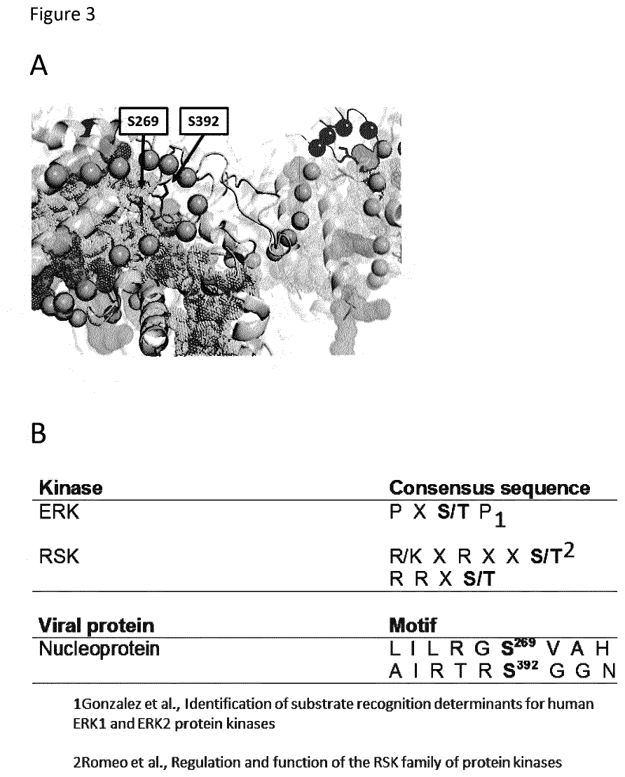

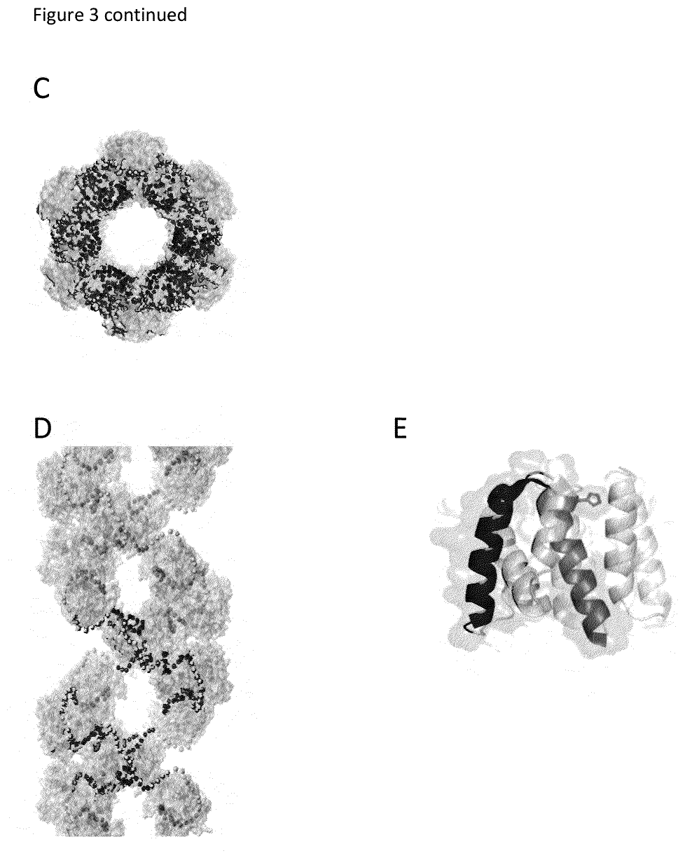

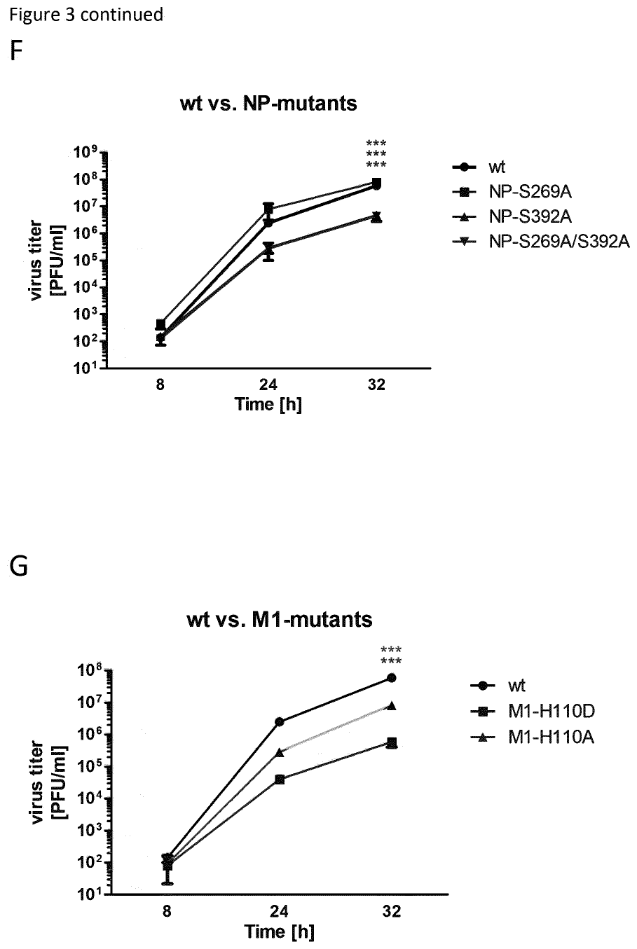

Under physiological conditions the Raf/MEK/ERK kinase cascade transmits extracellular signals within the cell to promote cellular processes like proliferation and differentiation. The signal is transmitted via sequential phosphorylation of the protein kinases (Yoon, 2006). To analyse putative phosphorylation sites within the viral NP protein, HEK293T cells were infected with a recombinant WSN/H1N1 virus expressing a Strep-tagged PB2 protein (MOI 5) and treated 3 h p.i. (post infection) with DMSO (1%) or CI-1040 (10 μM). 7 h p.i. the vRNPs were purified out of a total protein lysate. Phosphorylation pattern of the DMSO control and the CI-1040 samples were analyzed by mass spectrometry. Two Serine residues with a decreased phosphorylation state after inhibition of the pathway with MEK inhibitor CI-1040 could be identified. The crystallographic structure of a vRNP with bound RNA revealed that the two Serine residues 269 and 392 are in close proximity to each other near the nuclear export signals (NES) and the RNA-binding groove of NP. A vRNA loop (vRNA represented by spheres) surrounds both amino acids. Furthermore S269 is located within the NES2 and S392 is located near the NES2 and NES3 of the nucleoprotein (FIG. 3A). This loop is directed towards the inside of the helical vRNP complex (FIG. 3). The cryo-electron reconstruction of a helical part of the influenza virus A/Wilson-Smith(WSN)/1933 (H1N1) ribonucleoprotein shown in FIG. 3C was obtained from Protein Data Bank (PDB) ID 4BBL (Arranz et al., 2012). The identified regions within the NP protein (LILRGS²⁶⁹V, AIRTRS³⁹²G) showed no similarity to the consensus target sequence of the Serine/Threonine-kinase ERK (Pro-Xaa-Ser/Thr-Pro) as shown in FIG. 3B, which provides a comparison of the ERK (Gonzalez et al., 1991) and RSK (Romeo et al., 2012) consensus sequences with the identified phosphorylation motifs. Therefore it is unlikely that the identified Serine residues are directly phosphorylated by the kinase ERK. The consensus sequences of the downstream kinase 90 kDa ribosomal S6 kinase (RSK) (Arg/Lys-Xaa_Arg-Xaa-Xaa-Ser/Thr; Arg-Arg-Xaa-Ser/Thr) showed higher identities to the identified NP regions (Romeo, 2012). WSN mutants with non-phosphorylatable amino acids (aa) at the positions 269 and 392 (S269A, S392A, S269A/S392A) were generated to study the importance of these residues on the viral life cycle. It should be taken into account, that phospho-mimicking mutants could not be rescued, indicating that permanent negative charges at these positions are not tolerated by the virus. As can be seen from FIG. 3D, the RNA-binding sites of the M1 protein revealed a comparable shape to the identified vRNP loop. A positively charged Histidin (H110) that could interact with the phosphorylated serine residues is depicted in the bottom line and shown in FIG. 3E. The crystallographic structure of the influenza virus A/Puerto Rico/8/1934 (H1N1) M1 protein N-terminal domain was obtained from Protein Data Bank (PDB) ID 1EA3 (Arzt et al., 2001). Structural comparison of the vRNA loop with the RNA-binding site of the M1 protein revealed a comparable shape with a positively charged Histidine. We hypothesize that this Histidine might interact with the negative charges of the phosphorylated Serine residues. WSN mutants mimicking neutral (H110A) and negative (H110D) charges were generated and the results are shown in FIGS. 3F and 3G, where the growth kinetics of constitutive non-phosphorylated WSN-NP (S269A, S392A, S269A/S392A) and WSN-M1 (H110D, H110A) mutants are presented. Here, A549 cells were infected with the different WSN virus mutants or the wildtype virus (MOI 0.01). 8 h, 24 h and 32 h p.i. progeny virus titers were determined by standard plaque assay. Data represents mean of three independent experiments. Each experiment was performed in triplicates. Statistical significance was evaluated by two-way ANOVA followed by Bonferroni post-test.

### Example 2

### RSK1 as a Link Between the Raf/MEK/ERK-Pathway and vRNP Export

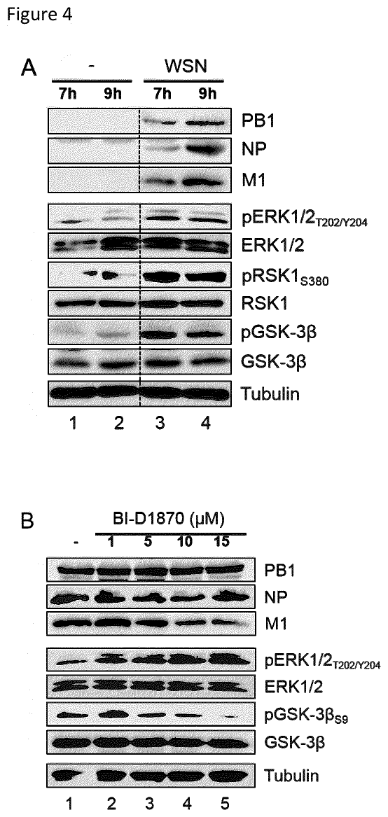

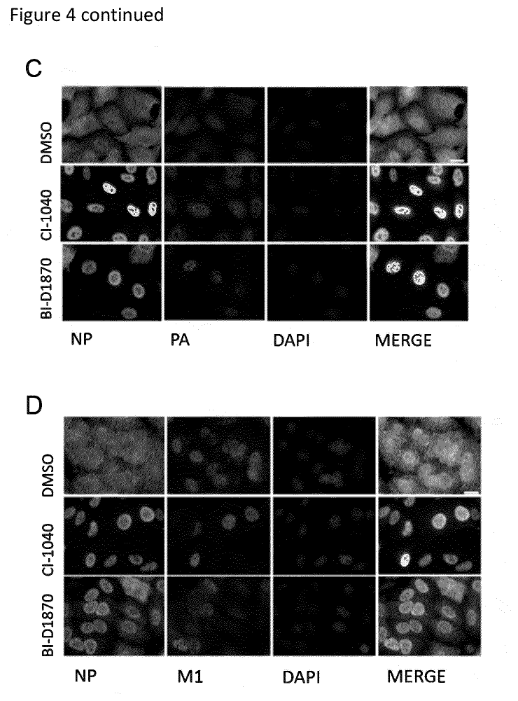

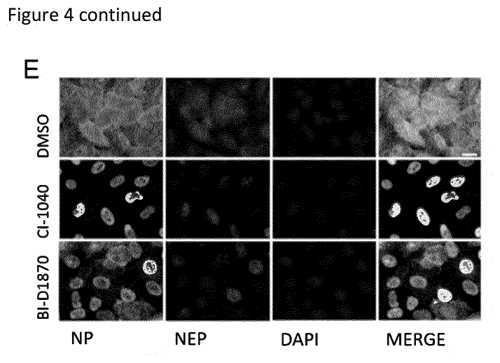

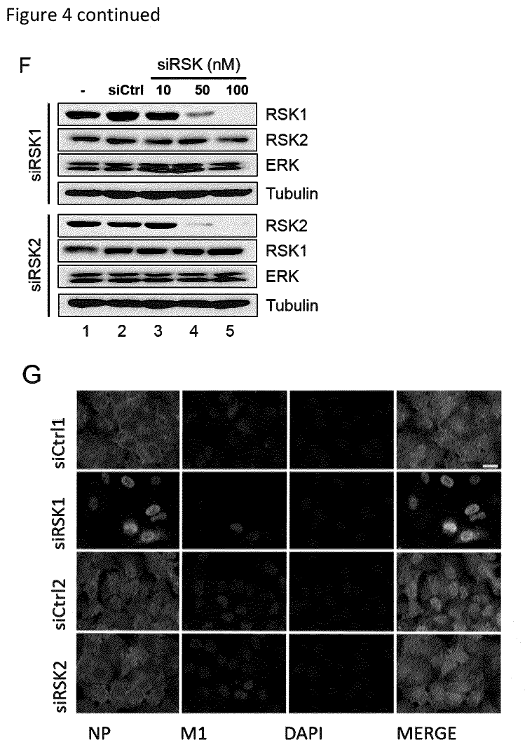

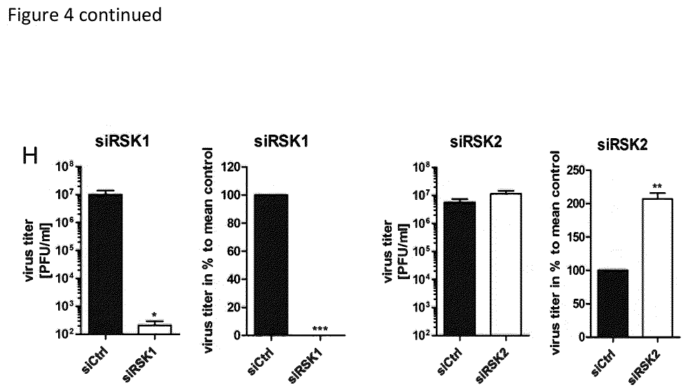

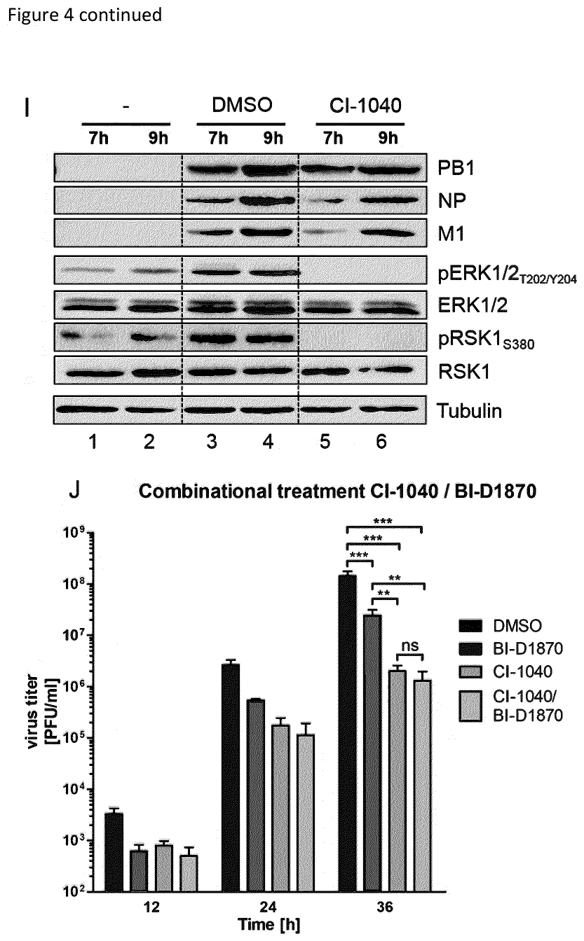

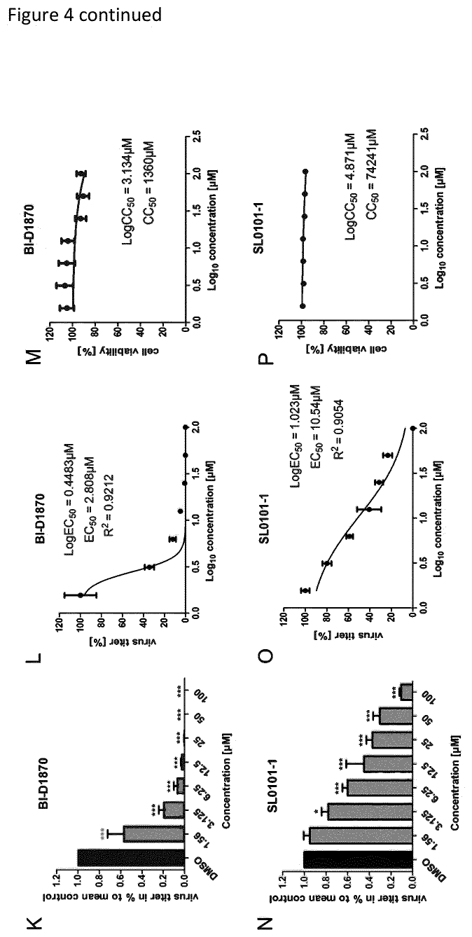

To shed light on the question whether RSK is the link between the activation of the Raf/MEK/ERK signaling pathway and the nuclear export of newly synthesized vRNPs, its activation during the viral life cycle was analyzed. This activation could be blocked by incubation with the MEK inhibitor CI-1040, indicating that the virus induced RSK activation is mediated by the Raf/MEK/ERK pathway. Specifically, A549 cells were infected with WSN/H1N1 (MOI 5). Cells were lysed 7 h and 9 h p.i. and cell lysates were used for western blot analysis of the phosphorylation state of ERK1/2, RSK1 and GSK-3β. Viral replication was determined by protein expression of PB1, NP and M1. Tubulin was detected as loading control. Results of one out of two independent experiments are shown in FIG. 4A. In the later stages of the infection not only ERK but also RSK and its downstream target GSK-3ß were phosphorylated and activated (FIG. 4A). In a further experiment, cells were treated with DMSO (1%) or indicated concentrations of BI-D1870 at 3 h p.i. Cells were lysed 7 h p.i. and cell lysates were used for western blot analysis of the phosphorylation state of ERK1/2 and GSK-3β. Viral replication was determined by protein expression of PB1, NP and M1. Tubulin was detected as loading control. Results of one out of three independent experiments are shown in FIG. 4B.

In a further experiment, cells were treated with DMSO (1%), CI-1040 (10 μM) or BI-D1870 (15 μM) 3 h p.i. and the results are shown in FIGS. 4C, 4D and 4E. 9 h p.i. cells were fixed and localization of vRNPs (NP-Alexa488) and (C) PA (Alexa-561), (D) M1 (Alexa-561) or (E) NEP (Alexa-561) was analyzed by epifluorescence microscopy. Nuclei were stained with Dapi. Epifluorescence microscopy pictures of single focal planes are shown as representative images of (C,D) three or (E) two independent experiments, with a scale bar representing 20 μM.

In a further experiment to exclude off-target effects of the RSK inhibitors, RSK1 and RSK2 were targeted by siRNA-mediated knockdown in A549 cells and the effect on the viral life cycle was analyzed as shown in FIG. 4F. Specifically, A549 cells were transfected with the indicated concentrations of siRNA against RSK1 or RSK2. Untransfected and control transfected cells served as negative controls. 48 h post-transfection (p.t.) the total protein amount of RSK1, RSK2 and ERK1/2 was determined by western blot analysis. Tubulin was detected as loading control. As shown in FIG. 4G, A549 cells on cover glasses were transfected using siRNA concentrations of 100 nM. 48 h p.t. cells were infected with WSN/H1N1 (MOI 5). Cells were fixed 9 h p.i. and localization of vRNPs (NP-Alexa488) and M1 (Alexa-561) was analyzed by epifluorescence microscopy. Nuclei were stained with Dapi. Epifluorescence microscopy pictures of single focal planes are shown as representative images of three independent experiments using a scale bar which represents 20 μm. The data show, that exclusively RSK1 knockdown, but not knockdown of RSK2 led to a retention of vRNPs in the nucleus of infected cells.

Further, as shown in FIG. 4H, a RSK1 or RSK2 knockdown was introduced in A549 cells using siRNA concentrations of 100 nM. 48 h p.t. cells were infected with WSN/H1N1 (MOI 0.01). Progeny virus titers were determined 24 h p.i. by standard plaque assay. Titers of control siRNA were set to 100%. In addition, absolute viral titers are depicted in PFU/ml. Shown are means±SD of three independent experiments. Each experiment was performed in triplicates. Statistical significance was analyzed by paired two-tailed t-test (*p≤0.05; **p≤0.01, ***p≤0.001). Consistent with the effects of knockdown on the nuclear export it was found that within the multi replication analysis the RSK1 knockdown had an anti viral effect, shown by the decrease of viral titers. The RSK2 knockdown however seemed to have a slight proviral effect. This supportive effect of the RSK2 knockdown on the viral replication was already described by Kakugawa et al., 2009. This points out that the two RSK subtypes RSK1 and RSK2 have different roles within the influenza virus life cycle.

In a further Experiment, A549 cells were infected with WSN/H1N1 (MOI 5). 3 h p.i. cells were treated with DMSO (1%) or CI-1040 (10 μM). Untreated cells served as negative control. Cells were lysed 7 h and 9 h p.i. and cell lysates were used for western blot analysis of the phosphorylation state of ERK1/2 and RSK1. Viral replication was determined by protein expression of PB1, NP and M1. Tubulin was detected as loading control. Results of one out of three independent experiments are shown in FIG. 4I. Furthermore, an increase of the ERK activation after the inhibition of RSK was found, which can be explained by the inhibition of a negative feedback loop that under normal conditions would prevent the overactivation of the pathway (FIG. 4I).

In another experiment shown in FIG. 4J, A549 cells were infected with WSN/H1N1 (MOI 0.01). After infection cells were treated with BI-D1870 (10 μM), CI-1040 (10 μM), a combination of both inhibitors (each 10 μM) or solvent control DMSO (0.2%). 24 h p.i. progeny virus titers were analyzed by standard plaque assay. Data represents mean of four independent experiments. Each experiment was performed in duplicates. Statistical significance was evaluated by two-way ANOVA followed by Bonferroni post-test (ns p>0.05; **p≤0.01, ***p≤0.001). It was shown that there were no significant differences in progeny viral titers between the CI-1040 treatment and the BI-D1870 and CI-1040 combinational treatment, further showing that MEK and RSK get sequentially activated within the same pathway, namely the virus induced Raf/MEK/ERK pathway (FIG. 4J).

The inhibition of RSK with the specific inhibitor BI-D1870 after virus infection led to a concentration dependent reduction of the GSK-3ß phosphorylation, confirming its inhibitory effect on the RSK activation during the viral life cycle. Increasing concentrations of the inhibitor BI-D1870 negatively affected the vRNP nuclear export. To analyze the antiviral effect of RSK inhibitors we used BI-D1870 as well as SL0101-1 and determined the progeny virus titers after 24 h treatment with increasing concentrations from 1.56 μM to 100 μM (FIGS. 4K, N). Both inhibitors reduced the viral titers but BI-D1870 (EC50=2.808 μM) showed a higher effectiveness against the influenza infection than SL0101-1 (EC50=10.54 μM) (FIGS. 4L, O). Specifically, A549 cells were infected with WSN/H1N1 (MOI 0.01). After infection cells were treated with the depicted concentrations of BI-D1870 (I), SL0101-1 (L) or DMSO (0.1%). 24 h p.i. progeny virus titers were analyzed by standard plaque assay. Titers of DMSO-treated cells were set to 100%. Data represents mean of three independent experiments. Each experiment was performed in triplicates. Statistical significance was evaluated by one-way ANOVA followed by Dunnett's multiple comparison test (***p≤0.001). The results are shown in FIGS. 4K and 4N). Furthermore, in FIGS. 4L and 4O, progeny virus titers from (I,L) were used to calculate EC₅₀ values. Finally, in FIGS. 4M and 4P, A549 cells were treated with depicted concentrations (L) of BI-D1870. 24 h p.i. cell viability and cell membrane integrity was measured with the LDH cytotoxicity assay. Data in combination with (E) were used to calculated CC₅₀ values.

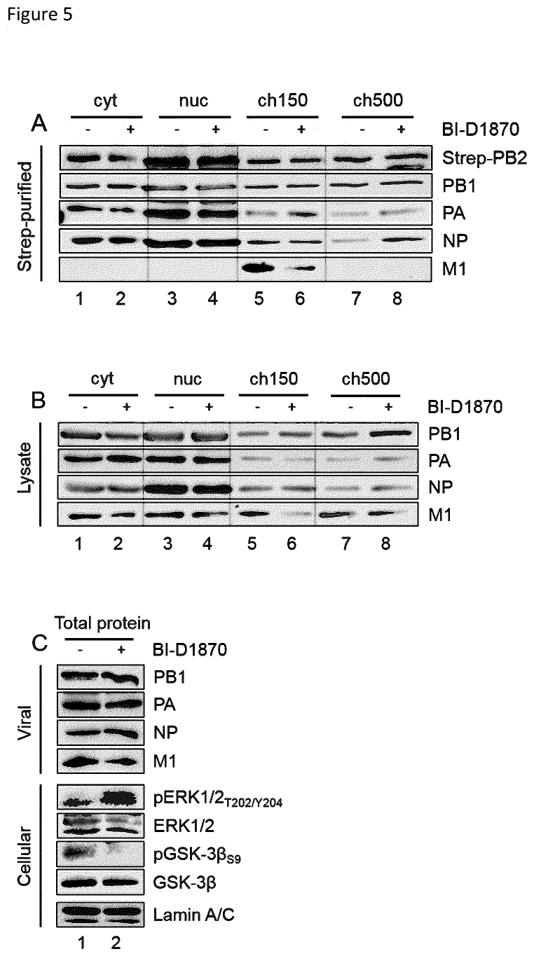

Chromatin fractionation assays after Strep-PB2-WSN virus infection and RSK inhibition were conducted to reveal the effect on the vRNP-M1 interaction. A549 cells were infected with Strep-PB2-WSN/H1N1 (MOI 5). 2.5 h post-infection (p.i.) cells were incubated with DMSO (1 %) or BI-D1870 (10 μM). 7 h p.i. cells were fractionated and the vRNP-complexes were purified out of the fractionated lysates by the PB2-Strep-Tag. The protein amounts of Strep-PB2, PB1, PA, NP and M1 were verified by western blot analysis. Results of one out of three independent experiments are shown. The BI-D1870 treatment led to comparable results with the CI-1040 inhibitor as shown in FIG. 5A. In FIG. 5B, Total protein amounts of the fractionated cell lysates from FIG. 5A were analysed by western blot. A decreased amount of M1 was co-purified with the Strep-PB2-vRNPs in the RSK inhibited samples, as well as higher protein amounts within the ch500 dense chromatin fraction (FIG. 5B). Additionally, protein amounts of total cell lysates from FIG. 5A were analysed by western blot. The phosphorylation state of ERK was analyzed by using an ERK specific antibody. The phosphorylation state of GSK-3β was analyzed by using a phospho-GSK and a GSK specific antibody. LaminA/C was detected as loading control. phospho-ERK blots in FIG. 5C shows the induction of the ERK1/2 phosphorylation state due to the inhibited negative feedback loop (FIG. 5C).

### Example 3

### Specific vRNP Export Block by MEK- and RSK-Inhibitors

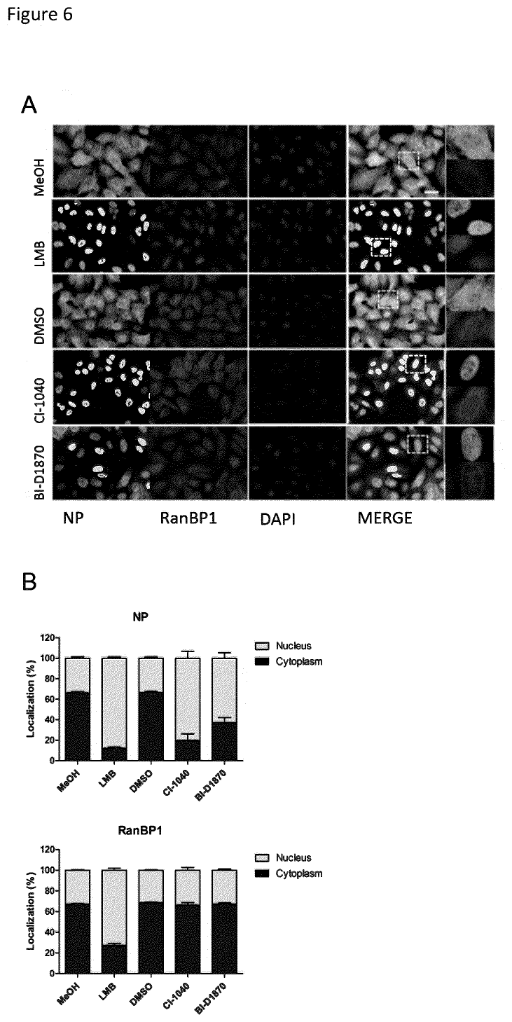

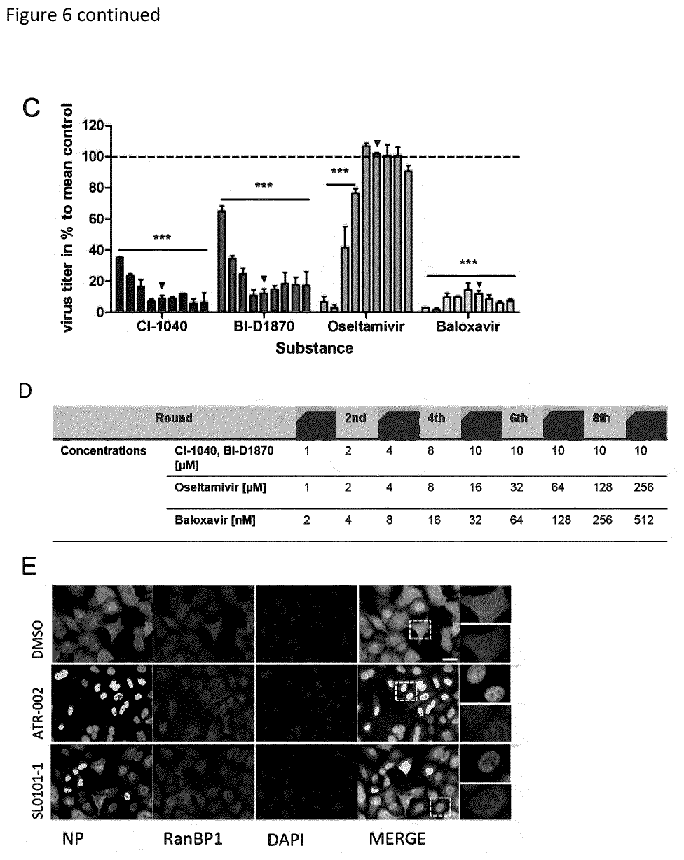

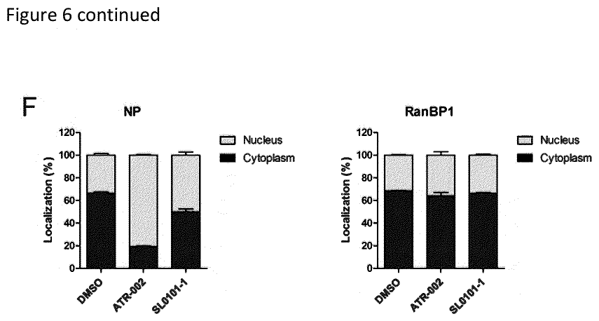

Leptomycin B broadly blocks the active nuclear export pathway by alkylating and inhibiting the major cellular export factor Crm1. This process is irreversible though cells inhibited will die. As a control for the cellular Crm1 export pathway we used the Ran binding protein RanBP1. With 23-kDa RanBP1 is small enough to diffuse through the nuclear pores within the nucleus. It was shown that a high nuclear concentration of RanBP1 is toxic for the cells. Therefore it has to be exported permanently back in the cytoplasm by the Crm1-pathway. Leptomycin B leads to a nuclear accumulation of RanBP1 and can be used as a control for the general block of the Crm1 export pathway (Plafker, 2000). We used the MEK inhibitors CI-1040 and ATR-002 and the RSK inhibitors BI-D1870 and SL0101-1 to examine their effect on the Crm1 pathway. Cells were infected with the influenza A/WSN (H1N1) virus and treated with the inhibitors. 9 h p.i. the immunofluorescence staining was conducted. Specifically, A549 cells were infected with WSN/H1N1 (MOI 5). 3 h p.i. cells were treated with MeOH (0.1%), Leptomycin B (LMB) (5 nM), DMSO (1%), CI-1040 (10 μM) or BI-D1870 (15 μM). 9 h p.i. cells were fixed and cellular localization of vRNPs (NP-Alexa488) and RanBP1 (Alexa561) was analyzed by epifluorescence microscopy as shown in FIG. 6A, where nuclei were stained with Dapi, dashed squares represent zoom-in areas and the scale bar represents 20μM. The quantificational analysis revealed a nuclear retention of the viral NP protein for all tested inhibitors. Approximately 1000 cells for each substance of FIG. 6A were analyzed and scored for the protein localization using the ImageJ “Intensity Ration Nuclei Cytoplasm Tool”. Results are depicted in FIG. 6B as means±SD of three independent experiments. The highest retention rates were found for Leptomycin B and the two MEK-inhibitors as shown in FIG. 6B. A nuclear retention of RanBP1 was only detected for the Leptomycin B treated samples, as can be seen from FIG. 6B. This indicates that the inhibition of the Raf/MEK/ERK/RSK pathway has no general effect on the Crm1 export pathway but is specifically directed against the export of the vRNPs.

To test for the development of resistance, multi passaging experiments were performed. As shown in FIGS. 6C and 6D, A549 cells were infected with WSN/H1N1 (MOI 0.01) and treated with CI-1040, BI-D1870, Oseltamivir or Baloxavir in increasing concentrations as outlined in FIG. 6D. 24 h p.i. supernatants were collected and progeny viral titers were determined by standard plaque assay. For the following rounds fresh A549 cells were infected with the supernatants (MOI 0.01) and incubated with increasing amounts of the substances. Data represents mean of two independent experiments±SD. Titers of DMSO control were set to 100%. Each experiment was performed in triplicates. Statistical significance was analyzed by two-way ANOVA followed by Bonferroni post-tests to compare each substance to DMSO (***p<0.001). As can be seen in FIG. 6C, viral titers of CI-1040 and BI-D1870 treated infected cells is decreasing upon passaging with increasing concentrations of the inhibitors and titers stayed on a low level in subsequent passages, indicating a high barrier towards development of resistance. This is in clear contrast to Oseltamivir, were virus titers increased dramatically already starting in passage 3. From passage 5 on titers were back to control levels, indicating a completely resistant virus population. Although Baloxavir did not show a similar behavior as Oseltamivir it has to be noted, that also here titers were increasing with increasing concentrations up to passage 6, which is the opposite tendency compared to CI-1040 or BI-D1870, indicating a slightly decreased sensitivity to the drug. In summary, CI-1040 and BI-D1870 exhibit a high barrier towards development of resistance in clear contrast to Oseltamivir which rapidly induced resistance virus variants

Additionally, in FIGS. 6E and 6F, A549 cells were infected with WSN/H1N1 (MOI 5). 3 h p.i. cells were treated with DMSO (1%), ATR-002 (150 μM) or SL0101-1 (100 μM). 9 h p.i. cells were fixed and cellular localization of vRNPs (NP-Alexa488) and RanBP1 (Alexa561) was analyzed by epifluorescence microscopy. Nuclei were stained with Dapi. Dashed squares represent zoom-in areas. Approximately 1000 cells for each substance were analyzed and scored for the protein localization using the ImageJ “Intensity Ration Nuclei Cytoplasm Tool”. Results are depicted as means±SD of three independent experiments. Scale bar: 20μM. The quantificational analysis revealed a nuclear retention of the viral NP protein for ATR-002 (MEK inhibitor PD-0184264) and SL0101. A nuclear retention of RanBP1 was not detected for ATR-002 and SL0101. This indicates that the specific inhibition of vRNP nuclear export, but not the general Crm1 mediated export, can be reproduced with other inhibitors pf MEK and RSK and is not limited to inhibitors CI-1040 or BI-D1870.

### Example 4

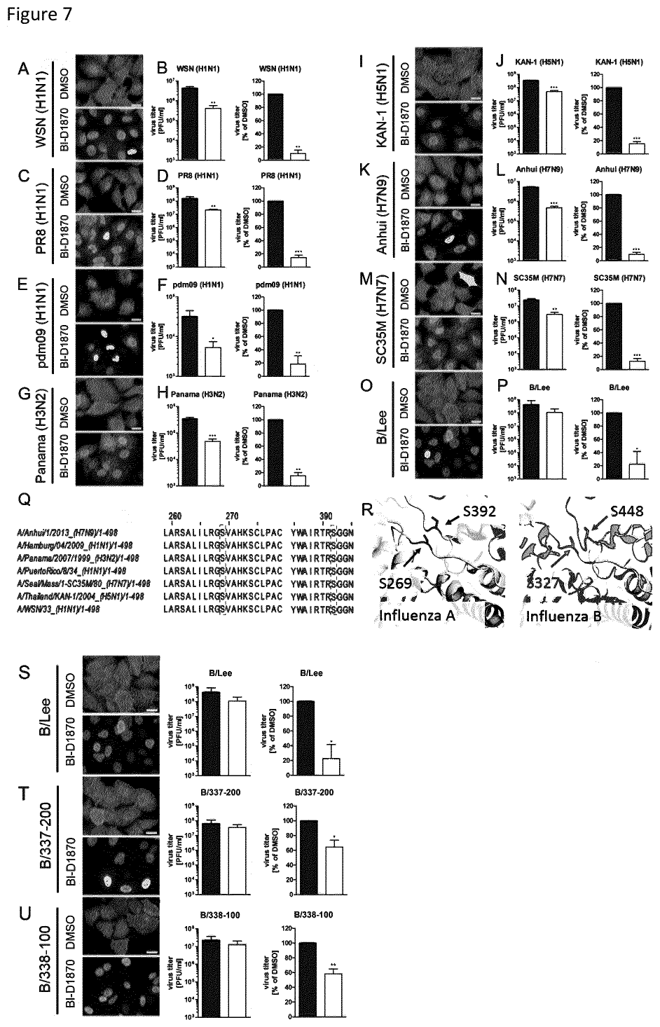

Broad anti-influenza activity of BI-D1870As the Raf/MEK/ERK/RSK-pathway is crucial for the influenza virus replication we used different influenza A subtypes, including a swine origin H1N1 pandemic virus, a seasonal H3N2 virus, different highly pathogenic avian influenza viruses, and an influenza B virus to determine the broad anti-viral activity (FIG. 7). Specifically, A549 cells (FIG. 7A-O) or MDCKII cells (FIG. 7P) were infected with human IAV (A-H) (WSN/H1N1, PR8M/H1N1, pdm09Hamburg/H1N1, Panama/H3N2), avian IAV (I-L) (KAN-1/H5N1, Anhui/H7N9), IAV/SC35M/H7N7 (M-N) or IBV (O-P) (B/Lee) (MOI 5 for immunofluorescence analysis (A,C,E,G,I,K,M,O), MOI 0.01 for multi replication cycle analysis (B,D,F,H,J,L,N,P)). 3 h p.i. cells were treated with BI-D1870 (15 μM) or DMSO (0.1%). (A, C, E, G, I, K, M) 9 h p.i. or (O) 12 h p.i. cells were fixed and localization of vRNPs (NP-Alexa488) was analyzed by epifluorescence microscopy. Nuclei were stained with Dapi. Epifluorescence microscopy pictures of single focal planes are shown (B,D,F,H,J,L,N,P). Directly after the infection cells were treated with BI-D1870 (15 μM) or DMSO (0.1%). 24 h p.i. progeny virus titers were analyzed by standard plaque assay. Data represents mean of three independent experiments. Titers of DMSO control were set to 100%. In addition, absolute viral titers are depicted in PFU/ml. Shown are means+SD of three independent experiments. Each experiment was performed in triplicates. Statistical significance was analyzed by paired two-tailed t-test (*p≤0.05; **p≤0.01, ***p≤0.001). Scale bar: 20 μm. In FIG. 7Q, a sequencial comparision of the NP regions containing S269 and S392 is shown for different viruses, indicating that the phosphorylation sites in NP are conserved among all these viruses. Sequences were obtained from Influenza Research Database and aligned by use of Jalview. In FIG. 7R, Crystallographic structures of the Influenza A/WSN (H1N1) and Influenza B/Lee NP regions revealed similiar structures. Influenza B NP S327 and S448 might represent comparable residues to Influenza A. The crystallographic structures were obtained from Protein Data Bank (Influenza virus A/Wilson-Smith/1933 (H1N1) ID: 4BBL; Arranz et al., 2012; Influenza virus B/HongKong/CUHK-24964/2004 ID: 3TJ0; Ng et al., 2012). As can be seen from FIG. 7, all tested viruses showed a nuclear retention of newly synthesized vRNPs and a reduction of progeny viral titers of around 80 to 90%. These results clarify the dependence of the influenza viruses on the Raf/MEK/ERK/RSK pathway.

### Example 5

### Specific Nuclear Localization of RSK1 After the Influenza A Infection

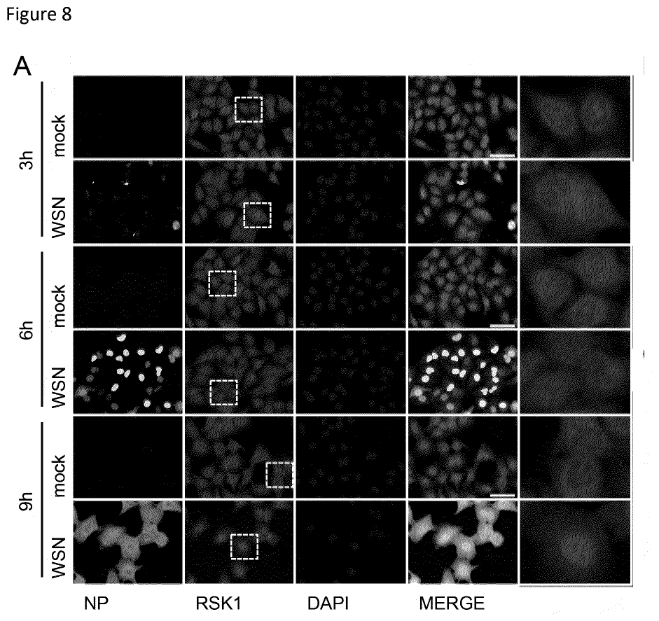

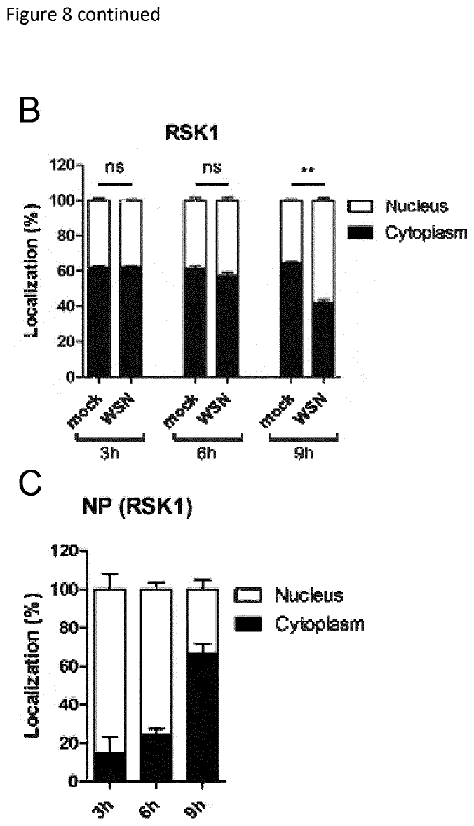

Former experiments revealed different contributions of the isoforms RSK1 and RSK2 to the viral life cycle. RSK2 has an anti-viral effect, whereas RSK1 obviously seems to act virus supportive. Upon its activation at the plasma membrane and the cytosol, RSK1 translocates into the nucleus. If RSK1 is the kinase phosphorylating NP, its cellular distribution should change during the viral life cycle, depending on the virus induced activation of the Raf/MEK/ERK pathway. To address this hypothesis A549 cells were either mock infected or infected with the WSN/H1N1 virus using a MOI of 5. 3 h, 6 h and 9 h p.i. the localization of NP and RSK1 was analyzed by immunofluorescence staining. Specifically, as shown in FIG. 8A, A549 cells were infected with WSN/H1N1 (MOI 5) or mock infected. After the indicated time points the cells were fixed and cellular localization of vRNPs (NP-Alexa-488) and RSK1 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. FIGS. 8 B and C show quantification of the cellular localization of NP and RSK1 from FIG. 8A. 10 epifluorescence microscopy pictures of each sample were analyzed in terms of the NP and RSK1 localization using the ImageJ “Intensity Ratio Nuclei Cytoplasm Tool”. Results are depicted as means±SD of three independent experiments. Statistical significance was analyzed by unpaired t-test with Welch's correction for each time point separately (ns p>0.05; **p≤0.01).As expected in the later time points of the infection an increase in the nuclear localization of RSK1 can be seen in FIG. 8A. The quantification of three independent experiments revealed a significant change in the nuclear concentration of RSK1 from 35.58%±0.59% for the mock infection to 57.73%±1.47% for the virus infection after 9 h (FIG. 8B), especially when the NP export took place (FIG. 8A,C). Indicating that the virus induces the nuclear import of RSK1.

### Example 6

### No Specific Nuclear Localization of RSK2 After the Influenza A Infection

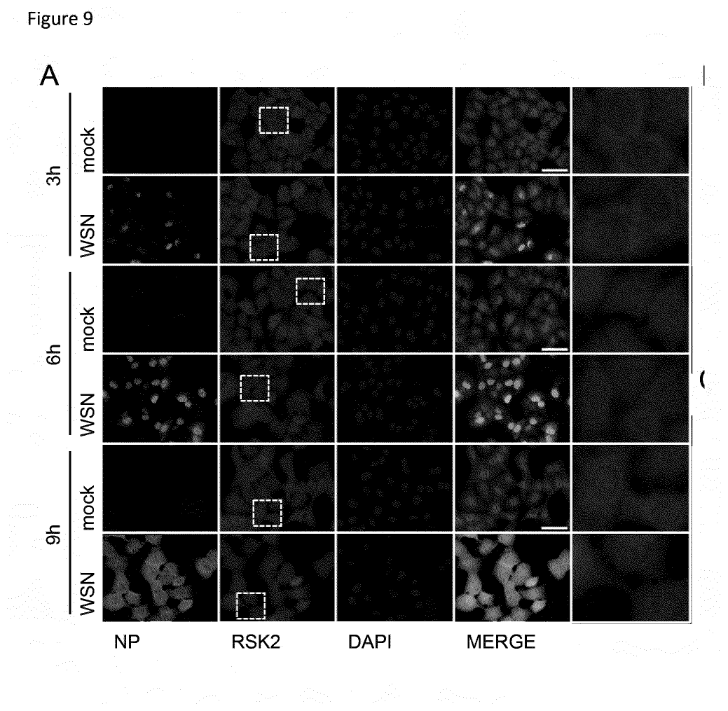

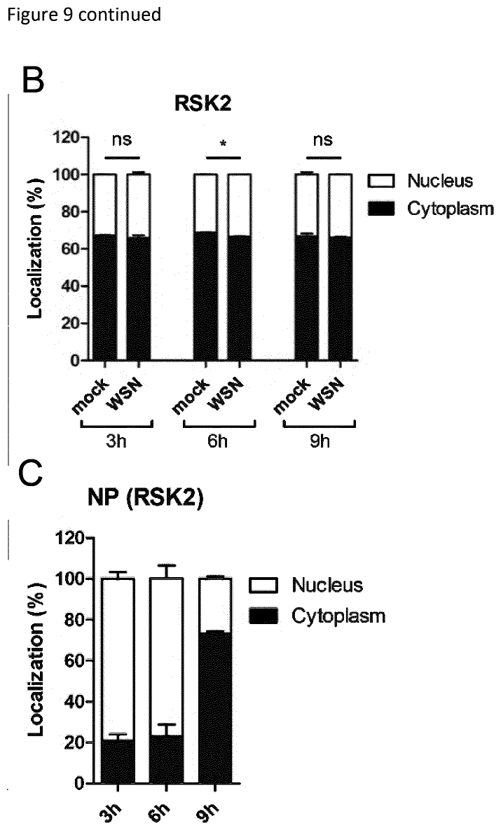

After it was confirmed that RSK1 enters the nucleus during the viral life cycle, the question was addressed whether virus induced activation of the Raf/MEK/ERK pathway would result in a nuclear localization of the anti-viral RSK2. Generally, the activation of the Raf/MEK/ERK-pathway results in the translocation of RSK1 and RSK2 into the nucleus. Therefore, the same experiment as described in Example 5 was conducted but the localization of RSK2 was analyzed. Specifically, as shown in FIG. 9A, A549 cells were infected with WSN/H1N1 (MOI 5) or mock infected. After the indicated time points the cells were fixed and cellular localization of vRNPs (NP-Alexa-488) and RSK2 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. FIGS. 9B, C show quantification of the cellular localization of NP and RSK2 from FIG. 9A. 10 epifluorescence microscopy pictures of each sample were analyzed in terms of the NP and RSK2 localization using the ImageJ “Intensity Ratio Nuclei Cytoplasm Tool”. Results are depicted as means±SD of three independent experiments. Statistical significance was analyzed by unpaired t-test with Welch's correction for each time point separately (ns p>0.05; **p≤0.05).

In contrast to RSK1, no change in the subcellular distribution of RSK2 was found, independently of the time point during the viral infection (FIGS. 9A,B). This results show, that the virus specifically induces the translocation of the virus supportive RSK1 without influencing the cellular distribution of antiviral acting RSK2.

### Example 7

### No Effect on ERK 1/2 Localization After Influenza A Infection

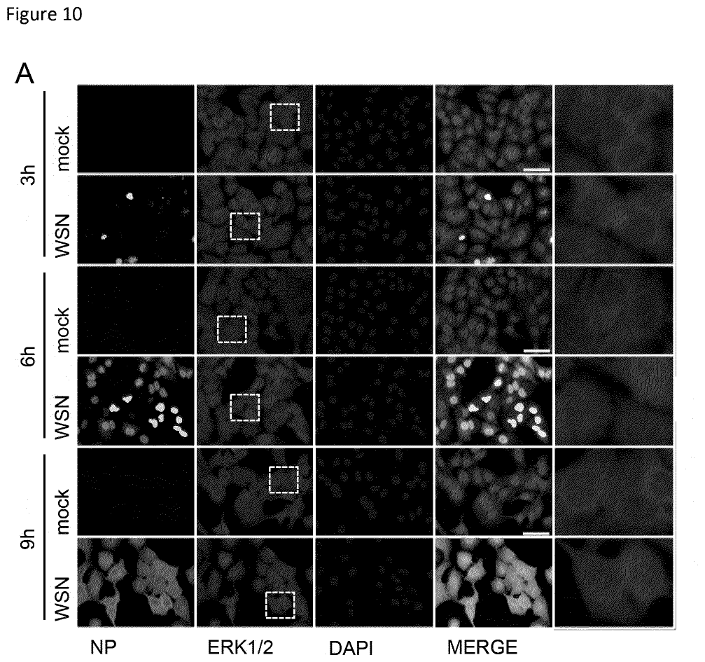

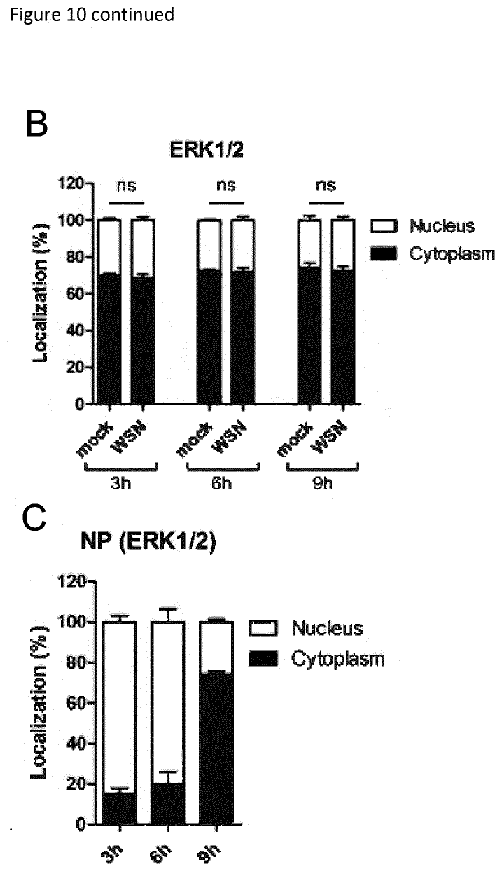

After the specific translocation of RSK1 was discovered, the question did arise whether the RSK upstream kinase ERK1/2 might enter the nucleus due to the viral pathway induction. Thus the same experiment as in Examples 5 and 6 was repeated with ERK. Specifically, as shown in FIG. 10A, A549 cells were infected with WSN/H1N1 (MOI 5) or mock infected. After the indicated time points the cells were fixed and cellular localization of vRNPs (NP-Alexa-488) and ERK1/2 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. FIGS. 10B, C show the quantification of the cellular localization of NP and ERK1/2 from FIG. 10A. 10 epifluorescence microscopy pictures of each sample were analyzed in terms of the NP and ERK1/2 localization using the ImageJ “Intensity Ratio Nuclei Cytoplasm Tool”. Results are depicted as means±SD of three independent experiments. Statistical significance was analyzed by unpaired t-test with Welch's correction for each time point separately (ns p>0.05). Surprisingly, no effect on the ERK1/2 localization can be seen after viral infection, independently of the analyzed time point (FIGS. 10A,B).

### Example 8

### Stimulation with TPA Results in Nuclear Localization of RSK2 and ERK1/2

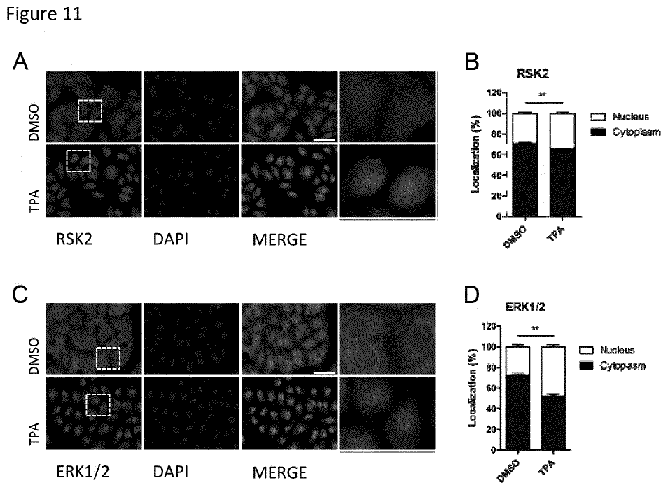

To exclude unspecific staining of RSK2 and ERK1/2 the Raf/MEK/ERK-pathway was stimulated with TPA. It is known that the incubation with TPA leads to the activation of the Raf/MEK/ERK-pathway resulting in the nuclear localization of ERK1/2 and RSKs within minutes after the stimulation. As shown in FIGS. 11A,C, A549 cells were stimulated with TPA (200 nM). The solvent DMSO served as negative control. After 1 h the cells were fixed and cellular localization of RSK2 or ERK1/2 (Alexa-561) was analyzed by epifluorescence microscopy. Nuclei were stained with Dapi. Dashed squares indicate zoom-in areas. Shown are representative epifluorescence microscopy pictures of single focal planes. Results of one out of three independent experiments are shown. Scale bar represents 50 μm. As shown in FIGS. 11B and D, Quantification of the cellular localization of RSK2 or ERK1/2 from 15 epifluorescence microscopy pictures of each sample were analyzed in terms of the RSK2 localization using the ImageJ “Intensity Ratio Nuclei Cytoplasm Tool”. Results are depicted as means±SD of three independent experiments. Statistical significance was analyzed by unpaired t-test with Welch's correction (**p≤0.01). Both kinases were found in the nuclei after an incubation with 100 nM TPA for 1 h, confirming that the staining is specific (FIGS. 11A,C). The quantification revealed nuclear accumulation of ERK1/2 from 27.99%±1.97% for the unstimulated samples to 48.02%±2.25% for the TPA-stimulated samples. The effect on the RSK2 kinase was not as prominent as for ERK1/2, with 29.37%±1.15% for the unstimulated samples to 34.89%±0.67% for the TPA-stimulated samples (FIGS. 11B,D).

### Example 9

### Long-Term Treatment with the MEK- or RSK-Inhibitors CI-1040 or BI-D1870 Does Not Introduce a Resistance in WSN/H1N1

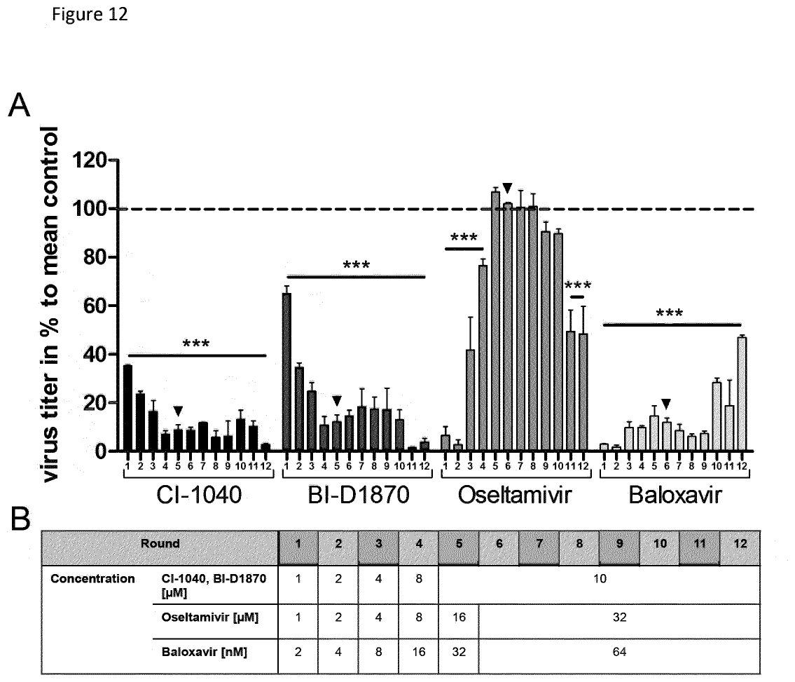

To compare the ability of antiviral drugs targeting the virus particle directly (Oseltamivir, Baloxavir) or acting antiviral via inhibition of the Raf/MEK/ERK/RSK pathway (CI-1040, BI-D1870) to induce resistance virus variants, A549 cells were infected with WSN/H1N1 (MOI 0.01) and treated with the MEK-inhibitor CI-1040, the RSK-inhibitor BI-D1870, the viral NA-inhibitor Oseltamivir acid or the viral cap-dependent endonuclease inhibitor of the polymerase subunit PA Baloxavir marboxil. 24 h p.i. progeny viral titers were determined by standard plaque titration. During the following rounds fresh A549 cells were infected with the collected supernatants (MOI 0.01) and treated with increasing concentrations of the different inhibitors. The rounds at which the concentrations remained constant are marked by arrowheads. The solvent DMSO (1%) served as negative control. Shown are relative viral titers. Titers of the DMSO control were arbitrary set to 100% (marked by dashed line). Data shown in FIG. 12A represents means±SD of two independent experiments. Each experiment was performed in triplicates. Statistical significance was calculated by two-way ANOVA followed by Bonferroni post-test (***p≤0.001). The inhibitor concentrations were increased to enhance the inhibitory effect and provoke the occurrence of mutations. The increasing concentrations of CI-1040 and BI-D1870 resulted in decreasing viral titers. Compared to the DMSO control, the titers were reduced in the first round using a 1 μM inhibitor concentration by 64.75%±0.32% and by 35.08%±3.20% for CI-1040 or BI-D1870, respectively. With increasing inhibitor concentrations, the titers did further reduce, up to round 4. At that point, both inhibitors were used in a concentration of 8 μM. From round 5 to round 12 CI-1040 and BI-D1870 were used with concentrations of 10 μM. Within round 5 to round 12 the average titer of the CI-1040 treatment was calculated with 8.45%±3.41% compared to the DMSO control. The average titer of the BI-D1870 treatment within round 5 to round 12 was calculated with 12.27%±6.36%. Neither a constant increase in the viral titers, nor a change in the plaque morphology was found for any time point during the 12 rounds, indicating that no resistance introducing mutation did occur. A complete resistance was found after 5 rounds of Oseltamivir treatment. At that point, an inhibitor concentration of 16 μM was used. At round 9 the titers started to decrease up to 48.30%±11.42%. This effect was accompanied by an increasing reduction in the plaque size. The average titer of the Baloxavir treatment from round 1 to round 9 was calculated with 8.08%±4.10%. A tendency in the titer increase started at round 10 with an average titer of 28.39%±1.88%. At round 12 the average titer further increased up to 46.94%±1.03% (FIG. 12A). FIG. 12B shows an overview of the inhibitor concentrations used for each round. At round 5 (CI-1040, BI-D1870) or round 6 (Oseltamivir, Baloxavir) the concentrations remained constant.

### REFERENCES

Adamantane resistance among influenza A viruses isolated early during the 2005-2006 influenza season in the United States [Journal]/Verf. Bright R. A., Shay, D. K., Shu, B., Cox, N. J., and Klimov, A. I., //JAMA.-2006.-295.-S.891-894.

Adamantane-resistant influenza A viruses in the world (1902-2013): Frequency and distribution of the M2 gene mutations [Journal]/Verf. Dong G., Peng, C., Luo, J., Wang, C., Han, L., Wu, B., Ji, G., He, H., //PLOSone.-2015.-10(3).-S. e0119115.

Antiviral activity of the MEK-inhibitor U0126 against pandemic H1N1v and highly pathogenic avian influenza virus in vitro and in vivo [Artikel]/Verf. Droebner K., Pleschka, S., Ludwig, S., Planz, O.,.-2011.-92.-S. 195-203.

Association of functional influenza viral proteins and RNAs with nuclear chromatin and sub-chromatin structure [Artikel]/Verf. Takizawa N., Watanabe, K., Nouno, K., Kobayashi, N., Nagata, K.,.-2006.-8.-S. 823-833.

Baloxavir Marboxil for uncomplicated influenza in adults and adolescents [Journal]/Verf. Hayden F. G., Sugaya, N., Hirotsu, N., Lee, N., de Jong, M. D., Hurt, A. C., Ishida, T., Sekino, H., Yamada, K., Portsmouth, S., Kawaguchi, K., Shishido, T., Arai, M., Tsuchiya, K., Uehara, T., and Watanabe, A., //N Engl J Med.-2018.-379.-S. 913-923.

Characterization of influenza virus variants induced by treatment with the endonclease inhibitor baloxavir marboxil [Journal]/Verf. Omoto S., Speranzini, V., Hashimoto, T., Noshi, T., Yamaguchi, H., Kawai, M., Kawaguchi, K., Uehara, T., Shishido, T., Naito, A., and Cusack S. //Scientific Reports.-2018.-S. 8:9633.

Chromatin docking and exchange activity enhancement of RCC1 by Histones H2A and H2B [Journal]/Verf. Nemergut M. E., Mizzen, C. A., Stukenberg, T., Allis, C. D., and Macara, I. G., //Science.-2001.-292(5521).-S. 1540-1543.

Comparison of the clinical effectiveness of zanamivir and lanamivir octanoate for children with influenza A(H3N2) and B in the 2011-2012 season [Journal]/Verf. Koseki N., Kaiho, M., Kikuta, H., Oba, K., Togashi, T., Ariga, T., Ishiguro, N., //Influenza Other Respir Viruses.-2014.-8(2).-S. 151-158.

Comparison of the clinical effectivness of Oseltamivir and Zanamivir against influenza virus infection in children [Journal]/Verf. Sugaya N., Tamura, D., Yamazaki, M., Ichikawa, M., Kawakami, C., Kawaoka, Y., and Mitamura, K., //Clin Infect Dis.-2008.-47(3).-S. 339-345.

Disruption of the virus-host cell interaction and cell signaling pathways as an anti-viral approach against influenza virus infection [Journal]/Verf. Ludwig S.,.-2011.-392.-S. 837-847.

Facilitated nucleocytoplasmic shuttling of the Ran binding protein RanBP1 [Journal]/Verf. Plafker K., and Macara, I. G., //Molecular and cellular biology.-2000.-10:Bd. 20.-S. 3510-3521.

Identification of substrate recognition determinants for human ERK1 and ERK2 protein kinases [Journal]/Verf. Gonzalez F. A., Raden, D. L., Davis, R. J., //J Biol Chem.-1991.-33:Bd. 266.-S. 22159-63.

Incidence of adamantane resistance among influenza A (H3N2) viruses isolated worldwide from 1994 to 2005: a cause of concern [Journal]/Verf. Bright R. A., Medina, M. J., Xu, X., Perez-Oronaz, G., Wallis, R. T., Davis, T. M., Povinelli, L., Cox, N. J., and Klimov, A. I.-2005.-S. 67338-2.

Influenza virus propagation is impaired by inhibition of the Raf/MEK/ERK signalling cascade. [Journal]/Verf. Pleschka S., Wolff, T., Ehrhardt, C., Hobom, G., Planz, O., Rapp, U. R., and Ludwig, S., //Nat Cell Biol.-2001.-3(3).-S. 301-305.

Influenza virus ribonucleoprotein complexes gain preferential acces to cellular export machinery through chromatin targeting [Journal]/Verf. Chase G. P., Rameix-Welti, M. A., Zvirbliene, A., Zvirblis, G., Götz, V., Wolff, T., Naffakh, N., and Schwemmle, M., //PLoS Pathogens.-2011.-7(9).-S. e1002187.

Influenza virus-specific RNA and protein syntheses in cells infected with temperature-sensitive mutants defective in the genome segment encoding nonstructural proteins [Journal]/Verf. Wolstenholme A. J., Barrett, T., Nichol, S. T., and Mahy, B. W. J., //J Virol.-1980.-35(1).-S. 1-7.

Influenza-virus-induced signaling cascades: targets for antiviral therapy? [Journal]/Verf. Ludwig S., Planz, O., Pleschka, S., and Wolff, T.,.-2003.-9(2).-S. 46-52.

Inhibition of nuclear export of ribonucleoprotein complexes of the influenza virus by leptomycin B [Journal]/Verf. Watanabe K., Takizawa, N., Katoh, M., Hoshida, K., Kobayashi, N., and Nagata, K., //Virus Research.-2001.-77(1).-S. 31-42.

Interaction of the influenza virus nucleoprotein with the cellular CRM1-mediated nuclear export pathway [Journal]/Verf. Elton D., Simpson-Holley, M., Archer, K., Medcalf, L., Hallam, R., McCauley, J., and Digard, P., //J Virol.-2001.-75(1).-S. 408-419.

Late activation of the Raf/MEK/ERK pathway is required for translocation of the respiratory syncytial virus F protein to the plasma membrane and efficient viral replication [Journal]/Verf. Preugschas H. F., Hrincius, E. R., Mewis, C., Tran, G. V. Q., Ludwig, S., and Ehrhardt, C., //Cellular microbiology.-2018.-S. e12955.

Lower clinical effectivness of Oseltamivir against influenza B contrasted with influenza A infection in children [Journal]/Verf. Sugaya N., Mitamura, K., Yamazaki, M., Tamura, D., Ichikawa, M., Kimura, K., Kawakami, C., Kiso, M., Ito, M., Hatakeyama, S., and Kawaoka, Y., //Clin Infect Dis.-2007.-44(2).-S. 197-202.

MEK inhibition impairs influenza B virus propagation without emergence of resistant variants. [Journal]/Verf. Ludwig S., Wolff, T., Ehrhardt, C., Wurzer, W. J., Reinhardt, J., Planz, O., and Pleschka, S., //FEBS Lett.-2004.-561(1-3).-S. 37-43.

Mitogen-activated protein kinase-activated kinase RSK2 plays a role in innate immune responses to influenza virus infection. [Journal]/Verf. Kakugawa S., Shimojima, M., Goto, H., Horimoto, T., Oshimori, N., Neumann, G., Yamamoto, T., Kawaoka, Y., //J Virol.-2009.-6:Bd. 83.-S. 2510-7.

Molecular characterization of the complete genome of human influenza H5N1 virus isolates from Thailand [Journal]/Verf. Puthavathana P., Auewarakul, P., Charoenying, P. C., Sangsiriwut, K., Pooruk, P., Boonak, K., Khanyok, R., Thawachsupa, P., Kijphati, R., and Sawanpanyalert, P. //Journal of General Virology.-2005.-S. 423-433.

Molecular mechanisms of influenza virus resistance to neuraminidase inhibitors [Journal]/Verf. Gubareva L. V. //Virus Research.-2004.-S. 199-203.

Nuclear export of influenza virus ribonucleoproteins: Identification of an export intermediate at the nuclear periphery [Artikel]/Verf. Ma K., Roy, A. M. M., Whittaker, G. R.,.-2001.-282.-S. 215-220.

Nuclear transport of influenza virus ribonucleoproteins: The viral matrix protein (M1) promotes export and inhibits import [Journal]/Verf. Martin K., and Helenius, A., //Cell.-1991.-67(1).-S. 117-130.

Overview of the 3rd isirv-antiviral group conference-advances in clinical management. [Journal]/Verf. Hurt A. C., Hui, D. S., Hay, A., and Hayden, F. G., //Influenza Other Respir Viruses.-2015.-9(1).-S. 20-31.

Regulated production of an influenza virus spliced mRNA mediated by virus-specific products [Journal]/Verf. Smith D. B., and Inglis, S. C.,.-1985.-4(9).-S. 2313-2319.

Regulation and function of the RSK family of protein kinases [Journal]/Verf. Romeo Y., Zhang, X., Roux, P. P., //Biochem J.-2012.-2:Bd. 441.-S. 553-69.

Role of the influenza virus M1 protein in nuclear export of viral ribonucleoproteins [Journal]/Verf. Bui M., Wills, E. G., Helenius, A., and Whittaker, G. R.,.-2000.-74(4).-S. 1781-1786.

Signal Transduction through MAP Kinase cascades [Journal]/Verf. Lewis T. S., Shapiro, P. S., and Ahn, N. G., //Advances in Cancer Research.-1998.-74.-S. 49-139.

The clinically approved MEK inhibitor Trametinib efficiently blocks influenza A virus propagation and cytokine expression [Artikel]/Verf. Schräder T., Dudek, S.E., Schreiber, A., Ehrhardt, C., Planz, O., Ludwig, S.,.-2018.-157.-S. 80-92.

The extracellular signal-regulated kinase: Multiple substrates regulate diverse cellular functions [Journal]/Verf. Yoon S., and Seger, R., //Growth Factors.-2006.-24(1).-S. 21-44.

The mechansim of resistance to favipiravir in influenza [Journal]/Verf. Goldhill D. H., to Velthuis, A. J. W., Fletcher, R. A., Langat, P., Zambon, M., Lackenby, A., and Barclay, W. S., //PNAS.-2018.-115(45).-S. 11613-11618.

The MEK-inhibitor CI-1040 displays a broad anti-influenza virus activity in vitro and provides a prolonged treatment window compared to standard of care in vivo [Journal]/Verf. Haasbach E., Muller, C., Ehrhardt, C., Schreiber, A., Pleschka, S., Ludwig, S., and Planz, O., //Antiviral Research.-2017.-142.-S. 178-184.

The MEK-inhibitor CI-1040 displays a broad anti-influenza virus activity in vitro and provides a prolonged treatment window compared to standard of care in vivo [Journal]/Verf. Haasbach E., Muller, C., Ehrhardt, C., Schreiber, A., Pleschka, S., Ludwig, S., Planz, O., //Antiviral Research.-2017.-142.-S. 178-184.

The nuclear export protein of H5N1 influenza A viruses recruits Matrix 1 (M1) protein to the viral ribonucleoprotein to mediate nuclear export. [Journal]/Verf. Brunotte L., Flies, J., Bolte, H., Reuther, P., Vreede, F., and Schwemmle, M., //J Biol Chem.-2014.-289(29).-S. 20067-77.

The potential impact of neuraminidase inhibitor resistant influenza [Journal]/Verf. Lackenby A., Thompson, C., and Democratis, J., //Current Opinion in Infectious Diseases.-2008.-21(6).-S. 626-638.

1. An RSK inhibitor for use in a method for the prophylaxis and/or treatment of a viral disease.

2. The RSK inhibitor for the use of claim 1, where the RSK inhibitor is selected from the group consisting of BI-D1870, SL0101-1, LJH685, LJ1308, BIX 02565, FMK and a selective RSK1 inhibitor or a derivative, metabolite or pharmaceutically acceptable salt thereof.

3. The RSK inhibitor for the use of claim 2, wherein the selective RSK1 inhibitor is a si-RNA, shRNA or mi-RNA that selectively targets Rsk1 mRNA corresponding to conserved regions of RSK1 having the amino acid sequence of SEQ ID NO:1 or an antibody that binds to conserved regions of RSK1 having the amino acid sequence of SEQ ID NO:1.

4. The RSK inhibitor for the use according to any preceding claim, wherein the viral disease is an infection caused by a negative strand RNA virus, preferably an influenza virus.

5. The RSK inhibitor for the use according to any preceding claim, wherein the viral disease is an infection caused by a positive strand RNA virus, preferably a corona virus causing respiratory tract infection.

6. The RSK inhibitor for the use according to claim 4, wherein the influenza virus is resistant to a neuramidase inhibitor selected from the group consisting of oseltamivir, oseltamivir phosphate, zanamivir, peramivir, or laninamivir or a pharmaceutically acceptable salt thereof, or an inhibitor of the viral polymerase complex selected form the group of Favipiravir, Baloxavir, Baloxavir phosphate, Baloxavir Marboxil, or Pimodivir or a pharmaceutically acceptable salt thereof.

7. The RSK inhibitor for the use according to claim 4 or 6, wherein the influenza virus is of type H1N1, H2N2, H3N2, H5N1, H5N6, H5N8, H6N1, H7N2, H7N7, H7N9, H9N2, H10N7, N10N8 or, or a different influenza A virus or an influenza B virus such as the Yamagata or Victoria type.

8. The RSK inhibitor for the use according to any one of the preceding claim, wherein the RSK inhibitor is administered in combination with a second antiviral agent.

9. The RSK inhibitor for the use according to claim 8, wherein the second antiviral agent is selected from the group consisting of neuramidase inhibitors, polymerase complex inhibitors, endonuclease inhibitors, hemaglutinin inhibitors, non-structural protein 1 inhibitors, nucleoprotein inhibitors and MEK inhibitors.

10. The RSK inhibitor for the use according to claim 9 wherein the neuraminidase inhibitor is selected from oseltamivir, oseltamivir phosphate, zanamivir, peramivir or laninamivir or a pharmaceutically acceptable salt thereof.

11. The RSK inhibitor for the use according to claim 9 wherein the polymerase complex inhibitor is baloxavir, Baloxavir phosphate, Baloxavir Marboxil, Favipiravir or Pimodivir or a pharmaceutically acceptable salt thereof.

12. The RSK inhibitor for the use according to claim 9 wherein the MEK inhibitor is selected from the group consisting of CI-1040, PD-0184264, PLX-4032, AZD6244, AZD8330, AS-703026, GSK-1120212, RDEA-119, RO-5126766, RO-4987655, PD-0325901, GDC-0973, TAK-733, PD98059 and PD184352 or a pharmaceutically acceptable salt thereof.

13. A selective RSK1 inhibitor or a derivative, metabolite or pharmaceutically acceptable salt thereof that binds to RSK1 having the amino acid sequence of SEQ ID NO: 1 or the nucleotide sequence of SEQ ID NO:2 and shows no or low binding affinity to RSK2 having the amino acid sequence of SEQ ID NO: 3 or the nucleotide sequence of SEQ ID NO:4, RSK3 having the amino acid sequence of SEQ ID NO: 5 or the nucleotide sequence of SEQ ID NO:6 and RSK4 having the amino acid sequence of SEQ ID NO: 7 or the nucleotide sequence of SEQ ID NO:8.

14. The selective RSK1 inhibitor of claim 13, wherein the inhibitor is a si-RNA, shRNA, mi-RNA, an antibody, or a small molecule.

15. A pharmaceutical composition comprising a selective RSK1 inhibitor according to claim 13 or 14 alone or in combination with a second antiviral agent for use in the prophylaxis and/or treatment of a viral disease.

16. Method of identifying specific RSK1 inhibitors comprising the steps of:
(i) Screening a library of potential inhibitors for binding to RSK1 having the amino acid sequence of SEQ ID NO: 1 or the nucleotide sequence of SEQ ID NO:2;
(ii) selecting the inhibitors that were found to bind RSK1 in step (i) and screening these for binding to RSK2 having the amino acid sequence of SEQ ID NO: 3 or the nucleotide sequence of SEQ ID NO:4, RSK3 having the amino acid sequence of SEQ ID NO: 5 or the nucleotide sequence of SEQ ID NO:6 and RSK4 having the amino acid sequence of SEQ ID NO: 7 or the nucleotide sequence of SEQ ID NO:8; and
(iii) selecting the inhibitors from step (ii) that do not bind to RSK2, RSK3 or RSK4 as specific RSK1 inhibitors.


SEQUENCE LISTING (8 sequences)

SEQ ID NO: 1 - protein, 735 aa, human
MPLAQLKEPWPLMELVPLDPENGQTSGEEAGLQPSKDEGVLKEISITHHVKAGSEKADPSHFELLKVLGQGSFGKVFLVRKVTRPDSGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHSHSADWWSYGVLMFEMLTGSLPFQGKDRKETMTLILKAKLGMPQFLSTEAQSLLRALFKRNPANRLGSGPDGAEEIKRHVFYSTIDWNKLYRREIKPPFKPAVAQPDDTFYFDTEFTSRTPKDSPGIPPSAGAHQLFRGFSFVATGLMEDDGKPRAPQAPLHSVVQQLHGKNLVFSDGYVVKETIGVGSYSECKRCVHKATNMEYAVKVIDKSKRDPSEEIEILLRYGQHPNIITLKDVYDDGKHVYLVTELMRGGELLDKILRQKFFSEREASFVLHTIGKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTPEEILTRIGSGKFTLSGGNWNTVSETAKDLVSKMLHVDPHQRLTAKQVLQHPWVTQKDKLPQSQLSHQDLQLVKGAMAATYSALNSSKPTPQLKPIESSILAQRRVRKLPSTTL

SEQ ID NO: 2 - DNA, 2208 bp, human
atgccgctcgcccagctcaaggagccctggccgctcatggagctagtgccgctggacccggagaatggacagacctcaggggaagaagctggacttcagccgtccaaggatgagggcgtcctcaaggagatctccatcacgcaccacgtcaaggctggctctgagaaggctgatccatcccatttcgagctcctcaaggttctgggccagggatcctttggcaaagtcttcctggtgcggaaagtcacccggcctgacagtgggcacctgtatgctatgaaggtgctgaagaaggcaacgctgaaagtacgtgaccgcgtccggaccaagatggagagagacatcctggctgatgtaaatcacccattcgtggtgaagctgcactatgccttccagaccgagggcaagctctatctcattctggacttcctgcgtggtggggacctcttcacccggctctcaaaagaggtgatgttcacggaggaggatgtgaagttttacctggccgagctggctctgggcctggatcacctgcacagcctgggtatcatttacagagacctcaagcctgagaacatccttctggatgaggagggccacatcaaactcactgactttggcctgagcaaagaggccattgaccacgagaagaaggcctattctttctgcgggacagtggagtacatggcccctgaggtcgtcaaccgccagggccactcccatagtgcggactggtggtcctatggggtgttgatgtttgagatgctgacgggctccctgcccttccaggggaaggaccggaaggagaccatgacactgattctgaaggcgaagctaggcatgccccagtttctgagcactgaagcccagagcctcttgcgggccctgttcaagcggaatcctgccaaccggctcggctccggccctgatggggcagaggaaatcaagcggcatgtcttctactccaccattgactggaataagctataccgtcgtgagatcaagccacccttcaagccagcagtggctcagcctgatgacaccttctactttgacaccgagttcacgtcccgcacacccaaggattccccaggcatcccccccagcgctggggcccatcagctgttccggggcttcagcttcgtggccaccggcctgatggaagacgacggcaagcctcgtgccccgcaggcacccctgcactcggtggtacagcaactccatgggaagaacctggtttttagtgacggctacgtggtaaaggagacaattggtgtgggctcctactctgagtgcaagcgctgtgtccacaaggccaccaacatggagtatgctgtcaaggtcattgataagagcaagcgggatccttcagaagagattgagattcttctgcggtatggccagcaccccaacatcatcactctgaaagatgtgtatgatgatggcaaacacgtgtacctggtgacagagctgatgcggggtggggagctgctggacaagatcctgcggcagaagttcttctcagagcgggaggccagctttgtcctgcacaccattggcaaaactgtggagtatctgcactcacagggggttgtgcacagggacctgaagcccagcaacatcctgtatgtggacgagtccgggaatcccgagtgcctgcgcatctgtgactttggttttgccaaacagctgcgggctgagaatgggctcctcatgacaccttgctacacagccaactttgtggcgcctgaggtgctgaagcgccagggctacgatgaaggctgcgacatctggagcctgggcattctgctgtacaccatgctggcaggatatactccatttgccaacggtcccagtgacacaccagaggaaatcctaacccggatcggcagtgggaagtttaccctcagtgggggaaattggaacacagtttcagagacagccaaggacctggtgtccaagatgctacacgtggatccccaccagcgcctcacagctaagcaggttctgcagcatccatgggtcacccagaaagacaagcttccccaaagccagctgtcccaccaggacctacagcttgtgaagggagccatggctgccacgtactccgcactcaacagctccaagcccaccccccagctgaagcccatcgagtcatccatcctggcccagcggcgagtgaggaagttgccatccaccaccctgtga

SEQ ID NO: 3 - protein, 740 aa, human
MPLAQLADPWQKMAVESPSDSAENGQQIMDEPMGEEEINPQTEEVSIKEIAITHHVKEGHEKADPSQFELLKVLGQGSFGKVFLVKKISGSDARQLYAMKVLKKATLKVRDRVRTKMERDILVEVNHPFIVKLHYAFQTEGKLYLILDFLRGGDLFTRLSKEVMFTEEDVKFYLAELALALDHLHSLGIIYRDLKPENILLDEEGHIKLTDFGLSKESIDHEKKAYSFCGTVEYMAPEVVNRRGHTQSADWWSFGVLMFEMLTGTLPFQGKDRKETMTMILKAKLGMPQFLSPEAQSLLRMLFKRNPANRLGAGPDGVEEIKRHSFFSTIDWNKLYRREIHPPFKPATGRPEDTFYFDPEFTAKTPKDSPGIPPSANAHQLFRGFSFVAITSDDESQAMQTVGVHSIVQQLHRNSIQFTDGYEVKEDIGVGSYSVCKRCIHKATNMEFAVKIIDKSKRDPTEEIEILLRYGQHPNIITLKDVYDDGKYVYVVTELMKGGELLDKILRQKFFSEREASAVLFTITKTVEYLHAQGVVHRDLKPSNILYVDESGNPESIRICDFGFAKQLRAENGLLMTPCYTANFVAPEVLKRQGYDAACDIWSLGVLLYTMLTGYTPFANGPDDTPEEILARIGSGKFSLSGGYWNSVSDTAKDLVSKMLHVDPHQRLTAALVLRHPWIVHWDQLPQYQLNRQDAPHLVKGAMAATYSALNRNQSPVLEPVGRSTLAQRRGIKKITSTAL

SEQ ID NO: 4 - DNA, 2223 bp, human
atgccgctggcgcagctggcggacccgtggcagaagatggctgtggagagcccgtccgacagcgctgagaatggacagcaaattatggatgaacctatgggagaggaggagattaacccacaaactgaagaagtcagtatcaaagaaattgcaatcacacatcatgtaaaggaaggacatgaaaaggcagatccttcccagtttgaacttttaaaagtattagggcagggatcatttggaaaggttttcttagttaaaaaaatctcaggctctgatgctaggcagctttatgccatgaaggtattgaagaaggccacactgaaagttcgagaccgagttcggacaaaaatggaacgtgatatcttggtagaggttaatcatccttttattgtcaagttgcattatgcttttcaaactgaagggaagttgtatcttattttggattttctcaggggaggagatttgtttacacgcttatccaaagaggtgatgttcacagaagaagatgtcaaattctacttggctgaacttgcacttgctttagaccatctacatagcctgggaataatttatagagacttaaaaccagaaaatatacttcttgatgaagaaggtcacatcaagttaacagatttcggcctaagtaaagagtctattgaccatgaaaagaaggcatattctttttgtggaactgtggagtatatggctccagaagtagttaatcgtcgaggtcatactcagagtgctgactggtggtcttttggtgtgttaatgtttgaaatgcttactggtacactccctttccaaggaaaagatcgaaaagaaacaatgactatgattcttaaagccaaacttggaatgccacagtttttgagtcctgaagcgcagagtcttttacgaatgcttttcaagcgaaatcctgcaaacagattaggtgcaggaccagatggagttgaagaaattaaaagacattcatttttctcaacgatagactggaataaactgtatagaagagaaattcatccgccatttaaacctgcaacgggcaggcctgaagatacattctattttgatcctgagtttactgcaaaaactcccaaagattcacctggcattccacctagtgctaatgcacatcagctttttcgggggtttagttttgttgctattacctcagatgatgaaagccaagctatgcagacagttggtgtacattcaattgttcagcagttacacaggaacagtattcagtttactgatggatatgaagtaaaagaagatattggagttggctcctactctgtttgcaagagatgtatacataaagctacaaacatggagtttgcagtgaagattattgataaaagcaagagagacccaacagaagaaattgaaattcttcttcgttatggacagcatccaaacattatcactctaaaggatgtatatgatgatggaaagtatgtgtatgtagtaacagaacttatgaaaggaggtgaattgctggataaaattcttagacaaaaatttttctctgaacgagaggccagtgctgtcctgttcactataactaaaaccgttgaatatcttcacgcacaaggggtggttcatagagacttgaaacctagcaacattctttatgtggatgaatctggtaatccggaatctattcgaatttgtgattttggctttgcaaaacagctgagagcggaaaatggtcttctcatgactccttgttacactgcaaattttgttgcaccagaggttttaaaaagacaaggctatgatgctgcttgtgatatatggagtcttggtgtcctactctatacaatgcttaccggttacactccatttgcaaatggtcctgatgatacaccagaggaaatattggcacgaataggtagcggaaaattctcactcagtggtggttactggaattctgtttcagacacagcaaaggacctggtgtcaaagatgcttcatgtagaccctcatcagagactgactgctgctcttgtgctcagacatccttggatcgtccactgggaccaactgccacaataccaactaaacagacaggatgcaccacatctagtaaagggtgccatggcagctacatattctgctttgaaccgtaatcagtcaccagttttggaaccagtaggccgctctactcttgctcagcggagaggtattaaaaaaatcacctcaacagccctgtga

SEQ ID NO: 5 - protein, 733 aa, human
MDLSMKKFAVRRFFSVYLRRKSRSKSSSLSRLEEEGVVKEIDISHHVKEGFEKADPSQFELLKVLGQGSYGKVFLVRKVKGSDAGQLYAMKVLKKATLKVRDRVRSKMERDILAEVNHPFIVKLHYAFQTEGKLYLILDFLRGGDLFTRLSKEVMFTEEDVKFYLAELALALDHLHSLGIIYRDLKPENILLDEEGHIKITDFGLSKEAIDHDKRAYSFCGTIEYMAPEVVNRRGHTQSADWWSFGVLMFEMLTGSLPFQGKDRKETMALILKAKLGMPQFLSGEAQSLLRALFKRNPCNRLGAGIDGVEEIKRHPFFVTIDWNTLYRKEIKPPFKPAVGRPEDTFHFDPEFTARTPTDSPGVPPSANAHHLFRGFSFVASSLIQEPSQQDLHKVPVHPIVQQLHGNNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDVLCTITKTMDYLHSQGVVHRDLKPSNILYRDESGSPESIRVCDFGFAKQLRAGNGLLMTPCYTANFVAPEVLKRQGYDAACDIWSLGILLYTMLAGFTPFANGPDDTPEEILARIGSGKYALSGGNWDSISDAAKDVVSKMLHVDPHQRLTAMQVLKHPWVVNREYLSPNQLSRQDVHLVKGAMAATYFALNRTPQAPRLEPVLSSNLAQRRGMKRLTSTRL

SEQ ID NO: 6 - DNA, 2202 bp, human
atggacctgagcatgaagaagttcgccgtgcgcaggttcttctctgtgtacctgcgcaggaagtcgcgctccaagagctccagcctgagccggctcgaggaagaaggtgtcgtgaaggagatagacatcagccatcatgtgaaggagggctttgagaaggcagatccttcccagtttgagctgctgaaggttttaggacaaggatcctatggaaaggtgttcctggtgaggaaggtgaaggggtccgacgctgggcagctctacgccatgaaggtccttaagaaagccaccctaaaagttcgggaccgagtgagatcgaagatggagagagacatcttggcagaagtgaatcaccccttcattgtgaagcttcattatgcctttcagacggaaggaaagctctacctgatcctggacttcctgcggggaggggacctcttcacccggctctccaaagaggtcatgttcacggaggaggatgtcaagttctacctggctgagctggccttggctttagaccatctccacagcctggggatcatctacagagatctgaagcctgagaacatcctcctggatgaagaggggcacattaagatcacagatttcggcctgagtaaggaggccattgaccacgacaagagagcgtactccttctgcgggacgatcgagtacatggcgcccgaggtggtgaaccggcgaggacacacgcagagtgccgactggtggtccttcggcgtgctcatgtttgagatgctcacggggtccctgccgttccaggggaaggacaggaaggagaccatggctctcatcctcaaagccaagctggggatgccgcagttcctcagtggggaggcacagagtttgctgcgagctctcttcaaacggaacccctgcaaccggctgggtgctggcattgacggagtggaggaaattaagcgccatcccttctttgtgaccatagactggaacacgctgtaccggaaggagatcaagccaccgttcaaaccagcagtgggcaggcctgaggacaccttccactttgaccccgagttcacagcgcggacgcccacagactctcctggcgtccccccgagtgcaaacgctcatcacctgtttagaggattcagctttgtggcctcaagcctgatccaggagccctcacagcaagatctgcacaaagtcccagttcacccaatcgtgcagcagttacacgggaacaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgtcctgtgcaccatcaccaagaccatggactacctccattcccagggggttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggaggtcctgaagcgtcaaggctatgatgcggcgtgtgacatctggagtttggggatcctgttgtacaccatgctggcaggatttaccccttttgcaaatgggccagacgatacccctgaggagattctggcgcggatcggcagtgggaagtatgccctttctgggggaaactgggactcgatatctgacgcagctaaagacgtcgtgtccaagatgctccacgtggaccctcatcagcgcctgacggcgatgcaagtgctcaaacacccgtgggtggtcaacagagagtacctgtccccaaaccagctcagccgacaggacgtgcacctggtgaagggcgcgatggccgccacctactttgctctaaacagaacacctcaggccccgcggctggagcccgtgctgtcgtccaacctggctcagcgcagaggcatgaagagactcacgtccacgcggttgtag

SEQ ID NO: 7 - protein, 745 aa, human
MLPFAPQDEPWDREMEVFSGGGASSGEVNGLKMVDEPMEEGEADSCHDEGVVKEIPITHHVKEGYEKADPAQFELLKVLGQGSFGKVFLVRKKTGPDAGQLYAMKVLKKASLKVRDRVRTKMERDILVEVNHPFIVKLHYAFQTEGKLYLILDFLRGGDVFTRLSKEVLFTEEDVKFYLAELALALDHLHQLGIVYRDLKPENILLDEIGHIKLTDFGLSKESVDQEKKAYSFCGTVEYMAPEVVNRRGHSQSADWWSYGVLMFEMLTGTLPFQGKDRNETMNMILKAKLGMPQFLSAEAQSLLRMLFKRNPANRLGSEGVEEIKRHLFFANIDWDKLYKREVQPPFKPASGKPDDTFCFDPEFTAKTPKDSPGLPASANAHQLFKGFSFVATSIAEEYKITPITSANVLPIVQINGNAAQFGEVYELKEDIGVGSYSVCKRCIHATTNMEFAVKIIDKSKRDPSEEIEILMRYGQHPNIITLKDVFDDGRYVYLVTDLMKGGELLDRILKQKCFSEREASDILYVISKTVDYLHCQGVVHRDLKPSNILYMDESASADSIRICDFGFAKQLRGENGLLLTPCYTANFVAPEVLMQQGYDAACDIWSLGVLFYTMLAGYTPFANGPNDTPEEILLRIGNGKFSLSGGNWDNISDGAKDLLSHMLHMDPHQRYTAEQILKHSWITHRDQLPNDQPKRNDVSHVVKGAMVATYSALTHKTFQPVLEPVAASSLAQRRSMKKRTSTGL

SEQ ID NO: 8 - DNA, 2238 bp, human
atgctaccattcgctcctcaggacgagccctgggaccgagaaatggaagtgttcagcggcggcggcgcgagcagcggcgaggtaaatggtcttaaaatggttgatgagccaatggaagagggagaagcagattcttgtcatgatgaaggagttgttaaagaaatccctattactcatcatgttaaggaaggctatgagaaagcagatcctgcacagtttgagttgctcaaggttcttggtcaggggtcatttggaaaggtttttcttgttagaaagaagaccggtcctgatgctgggcagctctatgcaatgaaggtgttaaaaaaagcctctttaaaagttcgagacagagttcggacaaagatggagagggatatactggtggaagtaaatcatccatttattgtcaaattgcactatgcctttcagactgaagggaaactgtacttaatactggattttctcaggggaggagatgttttcacaagattatccaaagaggttctgtttacagaggaagatgtgaaattctacctcgcagaactggcccttgctttggatcatctgcaccaattaggaattgtttatagagacctgaagccagaaaacattttgcttgatgaaataggacatatcaaattaacagattttggactcagcaaggagtcagtagatcaagaaaagaaggcttactcattttgtggtacagtagagtatatggctcctgaagtagtaaataggagaggccattcccagagtgctgattggtggtcatatggtgttcttatgtttgaaatgcttactggtactctgccatttcaaggtaaagacagaaatgagaccatgaatatgatattaaaagcaaaacttggaatgcctcaatttcttagtgctgaagcacaaagtcttctaaggatgttattcaaaaggaatccagcaaatagattgggatcagaaggagttgaagaaatcaaaagacatctgttttttgcaaatattgactgggataaattatataaaagagaagttcaacctcctttcaaacctgcttctggaaaaccagatgatactttttgttttgatcctgaatttactgcaaaaacacctaaagattctcccggtttgccagccagtgcaaatgctcatcagctcttcaaaggattcagctttgttgcaacttctattgcagaagaatataaaatcactcctatcacaagtgcaaatgtattaccaattgttcagataaatggaaatgctgcacaatttggtgaagtatatgaattgaaggaggatattggtgttggctcctactctgtttgcaagcgatgcatacatgcaactaccaacatggaatttgcagtgaagatcattgacaaaagtaagcgagacccttcagaagagattgaaatattgatgcgctatggacaacatcccaacattattactttgaaggatgtctttgatgatggtagatatgtttaccttgttacggatttaatgaaaggaggagagttacttgaccgtattctcaaacaaaaatgtttctcggaacgggaggctagtgatatactatatgtaataagtaagacagttgactatcttcattgtcaaggagttgttcatcgtgatcttaaacctagtaatattttatacatggatgaatcagccagtgcagattcaatcaggatatgtgattttgggtttgcaaaacaacttcgaggagaaaatggacttctcttaactccatgctacactgcaaactttgttgcacctgaggttcttatgcaacagggatatgatgctgcttgtgatatctggagtttaggagtccttttttacacaatgttggctggctacactccatttgctaatggccccaatgatactcctgaagagatactgctgcgtataggcaatggaaaattctctttgagtggtggaaactgggacaatatttcagacggagcaaaggatttgctttcccatatgcttcatatggacccacatcagcggtatactgctgaacaaatattaaagcactcatggataactcacagagaccagttgccaaatgatcagccaaagagaaatgatgtgtcacatgttgttaagggagcaatggttgcaacatactctgccctgactcacaagacctttcaaccagtcctagagcctgtagctgcttcaagcttagcccagcgacggagcatgaaaaagcgaacatcaactggcctgtaa